(19) United States
(12) Patent Application Publication
(10) Pub. No.: US 2026/0165877 A1
(43) Pub. Date: Jun. 18, 2026

(54) SYSTEMS, DEVICES, AND METHODS FOR REDUCING A TEMPERATURE OF A USER'S MOUTH AND SURROUNDING TISSUE

(71) Applicant: THE BOARD OF TRUSTEES OF THE LELAND STANFORD JUNIOR UNIVERSITY, Stanford, CA (US)

(72) Inventors: Elisabet Rosàs Canyelles, San Mateo, CA (US); Andréas N. Ward, San Leandro, CA (US); Alice Rachel Finkelstein, San Carlos, CA (US)

(21) Appl. No.: 19/389,786

(22) Filed: Nov. 14, 2025

Related U.S. Application Data
(63) Continuation of application No. PCT/US24/30875, filed on May 23, 2024.
(60) Provisional application No. 63/503,949, filed on May 23, 2023.
(60) Provisional application No. 63/606,942, filed on Dec. 6, 2023.

(51) Int. Cl. A61F 7/00

(52) U.S. Cl. CPC A61F 7/00; A61F 2007/0017; A61F 2007/0056

(57) ABSTRACT

Mouth-cooling devices for absorbing heat from a user's mouth prior to, during, and/or after receiving treatment with chemotherapy may assist with reducing incidences of and/or mitigating pain and discomfort caused by oral mucositis that may result from receiving chemotherapy treatments. The mouth-cooling devices may include a plurality of components sized, shaped, and/or configured to fit within a user's mouth to cool oral tissue from inside and/or outside the user's oral cavity. In some instances, the mouth-cooling devices may have a body configured to fit within the user's oral cavity proximate to tissue so that it may absorb heat from the tissue. The mouth-cooling devices may also have an external component coupled to the body. The external component may be sized, shaped, and/or configured to sit outside the user's mouth to absorb heat from the user's lips and/or cheeks.

### RELATED APPLICATIONS

This patent application is a continuation of International Application No. PCT/US2024/030875, filed May 23, 2024, which claims benefit of and priority to U.S. Provisional Patent Application No. 63/503,949 filed on 23 May 2023 and entitled “DEVICE FOR COOLING THE ORAL MUCOSA” and U.S. Provisional Patent Application No. 63/606,942 filed on 6 Dec. 2023 and entitled “INTRAORALCOOLING DEVICE FOR DECREASING ORAL CAVITY TEMPERATURE,” the entire disclosures of which are expressly incorporated by reference herein in their entireties.

### FIELD OF THE INVENTION

The present invention is a medical device configured to cool the oral cavity, which may prevent oral mucositis (e.g., inflammation and ulceration of oral mucosa or oral tissue) as may occur during, or following, certain medical treatments such as infusion with chemotherapy drugs and/or exposure to radiotherapy.

### BACKGROUND

Inflammation of oral mucosa, oral tissue, and formation of ulcers or mouth sores, which is sometimes referred to as “oral mucositis” (OM) is a painful and debilitating medical condition that may be caused by, for example, auto-immune conditions, infectious diseases (e.g., COVID and herpes), and/or may be a result of chemotherapy and/or radiotherapy. For example, OM affects over 40% of patients receiving chemotherapy for all types of cancer treatment and 90% of patients at high-risk of developing OM, such as those receiving treatment for head & neck cancer and/or receiving stem cell transplants because the chemotherapy damages the cells of the oral mucosa and tissue. OM causes significant pain and impairs normal oral functions (speaking, eating, swallowing), which can lead to malnutrition, dehydration, and/or infections. These complications cause a major decline in quality of life and health for affected populations, which often require additional treatments and, in particularly severe cases, hospitalization.

The current standard of care for treating OM is primarily palliative and supportive, aimed at providing pain relief (e.g., topical analgesia and/or opioid analgesics), reducing inflammation (e.g., benzydamine and/or honey), increasing nutritional intake (e.g., total parenteral nutrition (TPN) and/or nasogastric tube insertion), and/or decreasing infection risks (e.g., use of antibiotic mouthwashes). However, these treatments are often ineffective and do not prevent, or reduce a severity of, OM. At times, OM treatment requires changes in administration (e.g., dosage and/or frequency of infusion) of chemotherapy and/or radiation, which may disrupt cancer treatment and/or negatively impact cancer recovery outcomes.

Traditionally, the oral cryotherapy to prevent and/or mitigate OM is provided using ice chips and/or chilled water. However, use of ice chips and/or chilled water has poor patient adherence due to patient discomfort and side effects including chills, nausea, pain, and teeth hypersensitivity. Additionally, use of ice chips does not uniformly cool the oral mucosa, especially regions under the tongue and where the gingiva meets the interior of the oral cavity.

### SUMMARY

The mouth-cooling systems and devices disclosed herein may include a body configured for placement within a user's oral cavity and an exterior component configured for placement outside the oral cavity so that a surface thereof may contact, or be proximate to, the user's lips, skin, and/or cheeks. In some embodiments, the exterior component may be configured to sized and configured to reside outside the user's mouth when the mouth-cooling device is worn and extend from a left commissure of the user's lips to a right commissure of the user's lips. The body and/or exterior component may include one or more cavities within which a heat-absorbing material may be present and/or sealed therein. The heat-absorbing material and/or the med as a whole may be configured and/or cooled so that absorbs heat from the user's oral cavity, mouth, lips, skin, and/or cheeks. Additionally, or alternatively, the mouth-cooling device may be configured to induce vasoconstriction and/or reduce capillary blood flow within the user's mouth, lips, and/or cheeks when worn. Additionally, or alternatively, the mouth-cooling device may be configured to reduce a metabolism rate of cells (e.g., oral epithelial stem cells) within the user's mouth, lips, and/or cheeks when worn. Additionally, or alternatively, the mouth-cooling device may be configured to prevent or mitigate development of oral mucositis for the user as may occur before, during, or after the user receives chemotherapy treatment and/or radiotherapy treatment.

In some embodiments, the mouth-cooling device and/or a portion thereof may be malleable and/or configured to deform to be proximate to the user's mouth, lips, or skin so that it may, for example, conform to user anatomy.

At times, the med with the heat-absorbing material positioned within cavities thereof may be cooled by, for example, storage in a refrigeration device and/or exposure to a cooling/heat-absorbing substance like dry ice. Additionally, or alternatively, the heat-absorbing material may be cooled and/or refrigeration prior to placement within a cavity of the med. Prior to insertion within the user's mouth, the mouth-cooling device may be cooled to a temperature at, or below 37, 20, 18, 15, 13, 10, 8, 6, or 4 degrees Celsius and/or within a range of 4-25 degrees Celsius.

In some embodiments, the heat-absorbing material resident within a cavity of the external component may be communicated to a cavity within the body and vice versa. In this way, heat-absorbing material may be exchanged between one cavity and another to, for example, refresh and/or replace heat-absorbing material that may have warmed following use with new, cooled, heat-absorbing material.

In many instances, the heat-absorbing material may be a phase-change material configured to change phase upon absorption of heat from the user's mouth and/or may be biocompatible. Exemplary heat-absorbing material include one or more of saline, water, a hydrogel, encapsulated water particles, encapsulated saline particles, gelatin, a suspension of water and a thickening agent, a combination of water and methylcellulose, and/or an emulsion of water and a thickening agent. Exemplary thickening agents include, but are not limited to a cellulose ether, methylcellulose, hydroxypropyl methylcellulose, carboxymethyl cellulose, and/or xanthan gum.

The body may be comprise an outer reservoir, an inner reservoir, and a connecting portion. The outer reservoir may include an outer reservoir cavity, the outer reservoir may be shaped to extend between a superior oral vestibule cavity of the user and an inferior vestibule cavity of the user when worn with a front side of the outer reservoir may be configured to be placed proximate to the superior oral vestibule cavity and the inferior oral vestibule cavity and a back side may be configured to be proximate to a buccal side of the user's upper jaw and lower jaw when worn by the user (i.e., positioned within the user's oral cavity or mouth). The inner reservoir may be shaped to extend between a lingual side of the user's upper jaw and a lingual side of the user's lower jaw and abut the user's tongue when worn and may include an inner reservoir cavity positioned therein. The connecting portion may be configured to connect the inner reservoir and the outer reservoir and fit between the user's upper jaw and lower jaw (e.g., between the user's teeth) when worn.

In some embodiments, the mouth-cooling devices and systems disclosed herein may include an over-tongue extension with an over-tongue extension cavity positioned therein. The over-tongue extension may extend from an inner side of the inner reservoir and may be sized and configured to reside between the user's tongue and mouth roof (i.e., on top of the tongue) when worn and the extension cavity may be filled with heat-absorbing material configured to absorb heat from the user's tongue and mouth roof when the body is positioned within the user's mouth. Additionally, or alternatively, the mouth-cooling devices and systems disclosed herein may comprise an under-tongue extension with an extension cavity resident therein. The under-tongue extension may extending from an inner side of the inner reservoir and may be sized and configured to reside under the user's tongue when the body is positioned within the user's mouth.

In some embodiments, the body and/or exterior component may include an opening sized and configured to accept insertion of a plug therein. The plug may act to open, or expose the exterior component cavity, outer reservoir cavity and/or inner reservoir cavity seals so that, for example, heat-absorbing material may be added thereto and/or extracted therefrom and/or sealed within the exterior component cavity, outer reservoir cavity and/or inner reservoir cavity.

In some embodiments, the exterior component is configured to extend to, between and/or beyond a left buccal mucosa extension and a right buccal mucosa extension of the user's mouth when the body is positioned within the user's mouth. Additionally, or alternatively, the exterior component may be configured to extend to, between, and/or a left commissure of the user's mouth and a right commissure of the user's mouth when the body is positioned within the user's mouth. Additionally, or alternatively, the exterior component may be configured to cool the user's lips and skin proximate to the user's lips when the mouth-cooling device is worn. For example, the exterior component may be configured to cool the user's skin opposing at least one of a superior oral vestibule and an inferior oral vestibule of the user when the mouth-cooling device and/or lips is worn.

In some embodiments, the mouth-cooling device may be sized and configured to hold the mouth-cooling device in place within the user's mouth when the mouth-cooling device is worn. For example, wherein an interior side of the exterior component may include one or more retention features configured to hold the mouth-cooling device in place within the user's mouth when the mouth-cooling device is worn. Exemplary retention features include, but are not limited to, an extension, a plurality of extensions, a ridge, and a deformable member. In some cases, the retention feature may include a retention feature cavity filled with heat-absorbing material. Additionally, or alternatively, the exterior component may include comprises a handle and a decorative feature extending from and exterior (not user facing) side thereof.

In some embodiments, the exterior component may be removably attached to the body. In some of these embodiments, the body includes a first mating feature and the exterior component includes a second mating feature configured to cooperate with the first mating feature to removably attach the exterior component to the first mating feature.

The first mating feature may be, for example, a coupling configured to accept insertion of the second mating feature therein. The coupling may be, for example, a threaded coupling configured to accept insertion of the second mating feature therein, the second mating feature may include threads configured to align with corresponding treads of the treaded coupling so that the second mating feature may be screwed into first mating feature, thereby attaching the exterior component to the body. Additionally, or alternatively, the first mating feature may be configured to snap together with the second mating feature thereby attaching the exterior component to the body. Additionally, or alternatively, the first mating feature may be configured to be held in place relative to the second mating feature via at least one of a sealant, a sleeve, an elastic band, a zip tie, and a gasket.

In many instances, the mouth-cooling devices and systems disclosed herein are configured to tolerate cleaning and/or sanitation for repeated use. For example, the mouth-cooling devices and systems disclosed herein may be dishwasher safe and/or configured to tolerate sterilization via application of heat, steam, and/or a chemical sterilizing agent.

In some embodiments, the mouth-cooling devices and/or systems disclosed herein may include a base configured to fit within a user's mouth and/or oral cavity. The base may comprise an outer edge configured to be removably coupled to a cooling attachment, an inner reservoir may include an inner reservoir cavity, the inner reservoir may be shaped to extend between a lingual side of the user's upper jaw and lower jaw and abut the user's tongue when worn, and a connecting portion configured to connect the inner reservoir and the outer reservoir and fit between the user's upper jaw and lower jaw when worn. The cooling attachment may be removably coupled to the outer edge of the body, the cooling attachment may include a cooling attachment cavity and may be shaped to extend between a superior oral vestibule cavity of the user and an inferior vestibule cavity of the user when worn with a front side of the outer reservoir may be configured to be placed proximate to the superior oral vestibule cavity and inferior oral vestibule cavity. Heat-absorbing material may be resident within inner reservoir cavity and the cooling attachment cavity, the heat-absorbing material may be configured to absorb heat from inside and outside the user's mouth when the mouth-cooling device is positioned therein. The cooling attachment may include an opening sized and configured to accept insertion of a plug therein, wherein the plug seals the heat-absorbing material within the cooling attachment cavity and a volume of heat-absorbing material may be removed from and/or added to the cooling attachment cavity via removal of the plug from the opening.

The mouth-cooling devices and/or systems disclosed herein may be used by placing the body of any of the mouth-cooling devices disclosed herein within the mouth of a user and positioning the exterior portion outside the user's mouth proximate to the user's lips so that the body and the exterior portion absorb heat from the user's mouth, lips, and/or cheeks, reduce capillary blood flow within the user's mouth, lips, and/or cheeks, induce vasoconstriction within the user's mouth or lips, reduce a metabolism rate of cells within the user's mouth, lips, and/or cheeks, or prevent or mitigate development of oral mucositis for the user. In some embodiments, a temperature of the user's mouth, mouth-cooling device, and/or portion/component thereof may be measured and/or monitored and, when a temperature the user's mouth, mouth-cooling device, and/or portion/component thereof rises above a threshold temperature (e.g., 10, 15, or 20 degrees Celsius) a first mouth-cooling device may be removed from the user's mouth and a second one may be placed therein/thereon.

### BRIEF DESCRIPTION OF THE DRAWINGS

The present invention is illustrated by way of example, and not limitation, in the figures of the accompanying drawings in which:

FIG. 1A is a schematic diagram illustrating a top-perspective view of an exemplary mouth-cooling device, in accordance with some embodiments of the present invention;

FIG. 1B is a schematic diagram illustrating a cross-section view of a portion of the mouth-cooling device of FIG. 1A, in accordance with some embodiments of the present invention;

FIG. 2A is a schematic diagram illustrating a top view of another exemplary mouth-cooling device, in accordance with some embodiments of the present invention;

FIG. 2B is a schematic diagram illustrating a cross-section view of the mouth-cooling device of FIG. 2A, in accordance with some embodiments of the present invention;

FIG. 2C is a schematic diagram illustrating a perspective view of the mouth-cooling device of FIG. 2A, in accordance with some embodiments of the present invention;

FIG. 2D is a schematic diagram illustrating a mid-sagittal cross-section view a user with the mouth-cooling device of FIG. 2A correctly positioned (i.e., being worn) in his anatomy (e.g., mouth, lips, cheeks, etc.), in accordance with some embodiments of the present invention;

FIG. 3 is a schematic diagram illustrating a perspective view of another exemplary mouth-cooling device, in accordance with some embodiments of the present invention;

FIG. 4A is a schematic diagram illustrating a perspective view of an exemplary mouth-cooling device with a handle and/or reservoir extending from an exterior surface thereof, in accordance with some embodiments of the present invention;

FIG. 4B is a schematic diagram illustrating a sectioned, or cut-away, view of the exemplary mouth-cooling device of FIG. 4A with a handle and/or reservoir, in accordance with some embodiments of the present invention;

FIG. 4C is a schematic diagram illustrating a mid-sagittal cross-section view a user with the mouth-cooling device of FIG. 4A correctly positioned (i.e., being worn) within his anatomy (e.g., mouth, lips, cheeks, etc.), in accordance with some embodiments of the present invention;

FIG. 5A is a schematic diagram illustrating a perspective view of another exemplary mouth-cooling device when in an open, or unfolded, configuration, in accordance with some embodiments of the present invention;

FIG. 5B is a schematic diagram illustrating the mouth-cooling device of FIG. 5A when in a closed, or folded configuration, in accordance with some embodiments of the present invention;

FIG. 5C is a schematic diagram illustrating a cross-section view a user with the mouth-cooling device of FIG. 5A inserted into his mouth when arranged in a folded configuration as shown in FIG. 5B, in accordance with some embodiments of the present invention;

FIG. 6 is a schematic diagram illustrating an exemplary system that includes a mouth-cooling device in communication with a peristaltic pump, in accordance with some embodiments of the present invention;

FIG. 7 is a block diagram of a kit including a plurality of mouth-cooling devices and thermally insulating packaging, in accordance with some embodiments of the present invention;

FIG. 8A is a schematic diagram illustrating a top view of another exemplary mouth-cooling device, in accordance with some embodiments of the present invention;

FIG. 8B is a schematic diagram illustrating a back perspective view of the mouth-cooling device of FIG. 8A, in accordance with some embodiments of the present invention;

FIG. 8C is a schematic diagram illustrating a side perspective view of the mouth-cooling device of FIG. 8A, in accordance with some embodiments of the present invention;

FIG. 8D is a schematic diagram illustrating a cross-section view of the mouth-cooling device of FIG. 8A, in accordance with some embodiments of the present invention;

FIG. 8E shows the mouth-cooling device of FIGS. 8A-8D positioned in a user's mouth;

FIG. 9A is a schematic diagram illustrating a top view of a base for a mouth-cooling device, in accordance with some embodiments of the present invention;

FIG. 9B is a schematic diagram illustrating a side view of the base of FIG. 9A, in accordance with some embodiments of the present invention;

FIG. 9C is a schematic diagram illustrating a top view of a cooling attachment, in accordance with some embodiments of the present invention;

FIG. 9D is a schematic diagram illustrating a perspective view of the cooling attachment of FIG. 9C, in accordance with some embodiments of the present invention;

FIG. 9E is a schematic diagram illustrating a cross-section view of the cooling attachment of FIG. 9C, in accordance with some embodiments of the present invention;

FIG. 9F is a schematic diagram illustrating an assembly of the base of FIG. 9A and the cooling attachment of FIG. 9C, in accordance with some embodiments of the present invention;

FIG. 9G is a schematic diagram illustrating a rear-perspective of the assembly of FIG. 9F, in accordance with some embodiments of the present invention;

FIG. 9H is a schematic diagram illustrating a cross-section view of the assembly of FIG. 9F, in accordance with some embodiments of the present invention;

FIG. 10A is a schematic diagram illustrating a top view of an exemplary mouth-cooling device body, in accordance with some embodiments of the present invention;

FIG. 10B is a schematic diagram illustrating a perspective view of the mouth-cooling device body of FIG. 10A, in accordance with some embodiments of the present invention;

FIG. 10C is a schematic diagram illustrating a front view of the mouth-cooling device body of FIG. 10A, in accordance with some embodiments of the present invention;

FIG. 10D is a schematic diagram illustrating a cross-section view of the mouth-cooling device body of FIG. 10A, in accordance with some embodiments of the present invention;

FIG. 11A is a schematic diagram illustrating a front view of an exemplary fixed-shape exterior component and/or an exemplary deformable exterior component, in accordance with some embodiments of the present invention;

FIG. 11B is a schematic diagram illustrating a top view of the fixed-shape exterior component and/or deformable exterior component of FIG. 11A, in accordance with some embodiments of the present invention;

FIG. 11C is a schematic diagram illustrating a side view of the fixed-shape exterior component and/or deformable exterior component of FIG. 11A, in accordance with some embodiments of the present invention;

FIG. 11D is a schematic diagram illustrating a rear perspective view of fixed-shape exterior component and/or deformable exterior component, in accordance with some embodiments of the present invention;

FIG. 11E is a schematic diagram illustrating a cross-section view of fixed-shape exterior component, in accordance with some embodiments of the present invention;

FIG. 11F is a schematic diagram illustrating a side view of a deformable exterior component, in accordance with some embodiments of the present invention;

FIG. 11G is a schematic diagram illustrating a cross-section view of the deformable exterior component of FIG. 11F, in accordance with some embodiments of the present invention;

FIG. 12A is a schematic diagram illustrating an exploded view of an exemplary mouth-cooling device, in accordance with some embodiments of the present invention;

FIG. 12B is a schematic diagram illustrating an exploded view of another mouth-cooling device, in accordance with some embodiments of the present invention;

FIG. 12C is a schematic diagram illustrating an exploded view of another mouth-cooling device, in accordance with some embodiments of the present invention;

FIG. 12D is a schematic diagram illustrating an exploded view of another mouth-cooling device, in accordance with some embodiments of the present invention;

FIG. 13A is a schematic diagram illustrating a top view of a body for a mouth-cooling device, in accordance with some embodiments of the present invention;

FIG. 13B is a schematic diagram illustrating a front view of the body of FIG. 13A, in accordance with some embodiments of the present invention;

FIG. 13C is a schematic diagram illustrating a cross-section view of the body of FIG. 13A, in accordance with some embodiments of the present invention;

FIG. 14A is a schematic diagram illustrating a top view a mouth-cooling device that includes the body of FIG. 13A and the fixed deformable exterior component and/or deformable exterior component of FIGS. 11A-11G, in accordance with some embodiments of the present invention;

FIG. 14B is a schematic diagram illustrating a perspective view of the mouth-cooling device of FIG. 14A, in accordance with some embodiments of the present invention;

FIG. 14C provides a top view of assembly mouth-cooling device of FIG. 14A with an arrangement of reference lines superimposed thereon, in accordance with some embodiments of the present invention;

FIG. 14D provides a cross-section view of the mouth-cooling device of FIG. 14A with a fixed-shape external component, in accordance with some embodiments of the present invention;

FIG. 14E provides a cross-section view of the mouth-cooling device of FIG. 14A with a deformable-shape external component, in accordance with some embodiments of the present invention;

FIG. 15A is a schematic diagram illustrating a mid-sagittal cross-section view a user with the mouth-cooling device of 14A correctly positioned (i.e., being worn) in his anatomy (e.g., mouth, lips, cheeks, etc.), in accordance with some embodiments of the present invention;

FIG. 15B is a schematic diagram illustrating a front view the user wearing the mouth-cooling device of 14A, in accordance with some embodiments of the present invention; and

FIG. 15C is a schematic diagram illustrating a bottom perspective view the user wearing the mouth-cooling device of 14A, in accordance with some embodiments of the present invention.

Throughout the drawings, the same reference numerals, and characters, unless otherwise stated, are used to denote like features, elements, components, or portions of the illustrated embodiments. Moreover, while the user invention will now be described in detail with reference to the drawings, the description is done in connection with the illustrative embodiments. It is intended that changes and modifications can be made to the described embodiments without departing from the true scope and spirit of the user invention as defined by the appended claims.

### DETAILED DESCRIPTION

Oral cryotherapy is a mechanism for promoting vasoconstriction and/or reducing cellular metabolism within the mouth, which leads to decreased delivery of OM-causing chemotherapy drugs to the oral tissue and mucosa before, during, and/or after a user receives chemotherapy drugs and/or radiotherapy. In particular, cooling oral epithelial stem cells may slow down their growth rates so that, for example off-target effects of chemotherapy agents on the oral epithelial stem cells that may contribute to the development and/or severity of OM may be avoided. Additionally, or alternatively, cooling-mediated vasoconstriction of the blood vessels of the oral tissue may reduce the blood flow to the oral tissue, which reduces delivery of chemotherapy agents to the mouth during treatment and, consequently, may reduce damage caused to the oral tissue caused by the chemotherapy agents.

In some embodiments, the mouth-cooling devices disclosed herein may be configured to cool a user's mouth and/or a portion thereof below 20 degrees Celsius and/or within a range of 4-25 degrees Celsius. In some embodiments, the mouth-cooling devices disclosed herein may be configured to preferentially cool regions of the mouth and/or oral tissue that may be at highest at risk for developing OM, such as an interior surface of the user's cheeks and/or lips, oral vestibules (crevices between the gums and lips) within the user's mouth, the user's soft palate mucosa, floor of the mouth, and/or the user's tongue (e.g., the ventral surface of the tongue), which may be collectively referred to herein as a “mouth.”

The mouth-cooling devices disclosed herein may be sized, shaped, and/or configured to fit within and/or conform to a user's mouth and cool one or more regions of the mouth to, for example, reduce cellular metabolism and/or promote vasoconstriction within the mouth at times from two sides (e.g., inside and outside the mouth).

In some embodiments, the mouth-cooling devices disclosed herein may be configured to cool a user's mouth, or a region thereof, for a pre-set duration of time that may correspond to, for example, a length of time the user receives a chemotherapy infusion (e.g., 15-60 minutes) and/or a period of time sufficient to slow cellular metabolism and/or induce vasoconstriction prior to, during, and/or after, for example, infusion with a chemotherapeutic agent and/or exposure to radiation. In some cases, a user may use a plurality of mouth-cooling devices in, for example, a sequential fashion to, for example, provide varying levels of cryotherapy to the mouth as, for example, a plurality (e.g., 2-5) of mouth-cooling devices that provide progressively stronger cryotherapy to gradually and/or sequentially reduce a temperature of the user's mouth from, for example, 37 to 25, 20, 15, 10, 5, or 4 degrees Celsius. Additionally, or alternatively, in some embodiments, a mouth-cooling device may be configured to provide consistent cryotherapy for a period of time (e.g., 10-20 minutes) and multiple mouth-cooling devices may be used sequentially to cool a user's mouth should a time period for cooling in excess of the period of time for which a single mouth-cooling device may provide the cryotherapy that is desired and/or needed. For example, if a 45 minutes of cryotherapy is recommended for a user, three mouth-cooling devices configured to provide cryotherapy may be sequentially used at 15 minute intervals to provide a total of 45 minutes of cryotherapy across the three mouth-cooling devices.

The mouth-cooling devices disclosed herein may comprise a body that comprises and/or houses a material capable of interfacing and/or thermally communicating with a user's mouth tissue (e.g., mucosa, lips, tongue, gingiva, etc.) to absorb heat therefrom. The body may be sized, shaped, and/or configured to be comfortably worn and/or prevent application of excessive friction and/or pressure to the user's mouth or a portion thereof. The body may be made from a soft, or deformable, non-toxic, material such as a food-grade polymer, food-grade silicone, a biocompatible elastomer, and/or a soft edible material (e.g., seaweed) that is selected and/or configured so that it does not significantly (e.g., remains functional) dissolve during use. The mouth-cooling device bodies described herein may be made via any manufacturing process such as 3D printing, injection molding, and/or cast molding.

In some cases, the mouth-cooling device bodies disclosed herein may be hollow, or partially hollow, and a volume of heat-absorbing material configured may be positioned within hollow openings, or cavities, so that it may extract heat from proximate mouth tissue. The heat-absorbing material may, or may not, be a phase change material and, in many cases, is non-toxic and/or biocompatible. Exemplary heat-absorbing materials include, but are not limited to, water, saline solution, encapsulated water particles, encapsulated saline particles, gelatin, a suspension, a hydrogel, and/or a thickened solution (e.g., a combination of water and a thickener such as a cellulose ester, methylcellulose, hydroxypropyl methylcellulose, carboxymethyl cellulose, and/or xanthan gum)). Additionally, or alternatively, the heat-absorbing material may be in gas and/or vapor form embodied as, for example, a vapor containing frozen saline droplets or particles that are steadily released into cavities within the mouth-cooling devices disclosed herein.

In some embodiments, the mouth-cooling device bodies disclosed herein may act and/or include as a thermal insulator configured to, for example, slow the heat transfer between heat-absorbing material and oral tissue, maintain thermal stability of the mouth-cooling device (e.g., keep surface temperature of the mouth-cooling device even and/or above a temperature threshold that causes pain or discomfort to a user), and/or prevent heat transfer from non-mucosal tissue within the mouth (e.g., teeth) that may be sensitive to cooling. Additionally, or alternatively, in some embodiments, the mouth-cooling devices disclosed herein may include an insulation layer positioned on an exterior surface, or a portion of an exterior surface thereof. The insulation layer may comprise, for example, a non-toxic and biocompatible material (e.g. silicone, foam, air, polyurethane, etc.).

In some embodiments, the mouth-cooling devices disclosed herein may be configured so that they comfortably fit into a user's mouth with no additional support or force needed to hold the mouth-cooling device in place. Additionally, or alternatively, one or more of the mouth-cooling devices disclosed herein may be configured to be self-contained so that it does not need to be connected to an additional piece of equipment to be functional during use.

In some embodiments, a user's mouth may be treated with, for example, an analgesic and/or pain relieving drug (e.g., lidocaine mouthwash) before, during, and/or after use with one or more of the mouth-cooling devices disclosed herein to, for example, reduce the user's sensitivity to the cryotherapy so that, for example, the user may tolerate the cryotherapy for a prescribed period of time. Additionally, or alternatively, a mouth-cooling device, or a portion thereof, may be coated and/or treated with an analgesic and/or pain relieving drug configured to interact with oral tissue during use of the mouth-cooling device.

In some embodiments, the mouth-cooling devices disclosed herein may include a temperature sensor (e.g., temperature sensors) and/or temperature indicating feature (e.g., non-toxic thermochromic inks or dyes embedded in the surface of the device that changes color responsively to detected temperature) configured to measure and/or provide an indication of the temperature inside a user's mouth and/or a region of the mouth (e.g., under the tongue). Temperature measurements and/or indications may be used by clinicians and/or caregivers to, for example, determine when to replace the mouth-cooling device (i.e., when the mouth-cooling device is no longer cooling to the desired temperature) and/or provide a feedback mechanism by which increase and/or decrease cooling provided by the mouth-cooling device.

In some embodiments, the mouth-cooling device may comprise a polymer with low softening point that may be moldable when, for example, heated. In these embodiments, the mouth-cooling device may be provided to a user in a general form (e.g., a curved shape) and upon application of heat to the device (e.g. in the microwave, or in a bath of hot water), it is introduced into the user's mouth, for example, the user, a clinician, and/or a professional caretaker and molded into a shape that approximates and/or mimics features of the user's mouth. As the mouth-cooling device returns to an unheated temperature, it retains the molded shape and is therefore customized to the user. In these embodiments, the mouth-cooling device(s) can be provided in a range of general forms that have varying initial general sizes and shapes configured to accommodate insertion into the mouth of different users, including adult and pediatric patients.

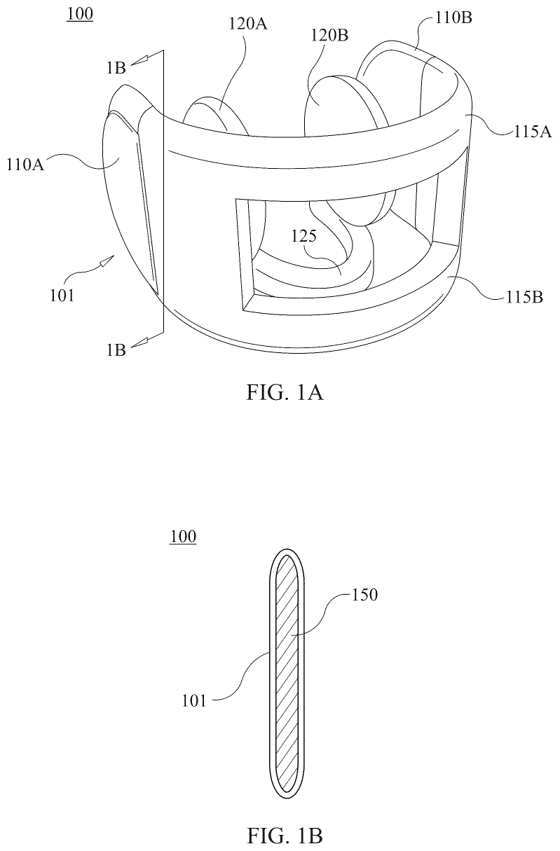

Turning now to the figures, FIG. 1A provides a top-perspective view of an exemplary mouth-cooling device 100 and FIG. 1B provides a cross-section view of a portion of mouth-cooling device 100 taken along sectioning line 1B-1B shown in FIG. 1A. Mouth-cooling device 100 includes a body 101, that may have one or more characteristics of the mouth-cooling device bodies disclosed herein. Body 101 includes a left buccal mucosa extension 110A, a right buccal mucosa extension 110B, a superior oral vestibule extension 115A, an inferior oral vestibule extension 115B, a left lateral tongue extension 120A, a right lateral tongue extension 120B, and a floor of mouth portion 125. Left and right buccal mucosa extensions 110A and 110B may be sized, positioned, and/or configured to be positioned on a respective left and right side of a user's mouth proximate to buccal mucosa (e.g., between the user's teeth and cheek) during use. Superior oral vestibule 115A may be sized, positioned, and/or configured to be positioned within a superior oral vestibule of a user's mouth between the gingiva of the user's superior jaw and superior lip (i.e., superior lip crevice during use). Inferior oral vestibule 115B may be sized, positioned, and/or configured to be positioned within an inferior oral vestibule of a user's mouth between the gingiva of the user's inferior jaw and inferior lip (i.e., inferior lip crevice) during use. Left and right lateral tongue extensions 120A and 120B may be sized, positioned, and/or configured to be positioned on a respective left and right side a user's tongue during use. Floor of mouth portion 125 may be sized, positioned, and/or configured to be positioned on a floor of a user's mouth under his or her tongue during use.

In some embodiments, mouth-cooling device 100 may be hollow (e.g., include one or more cavities) that may be filled with a heat-absorbing material as, for example, described herein and shown in FIG. 1B, which shows a cross-section of body 101 with a volume of heat-absorbing material 150, such as the heat-absorbing material disclosed herein, positioned therein.

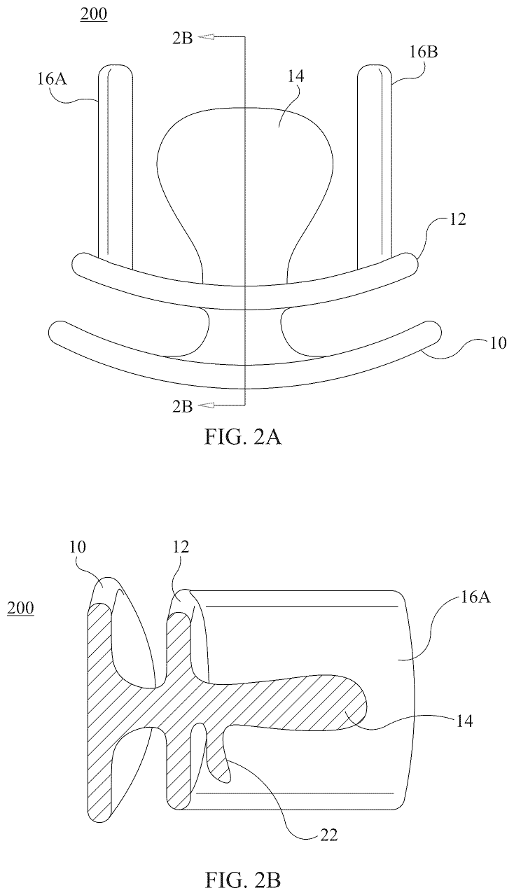

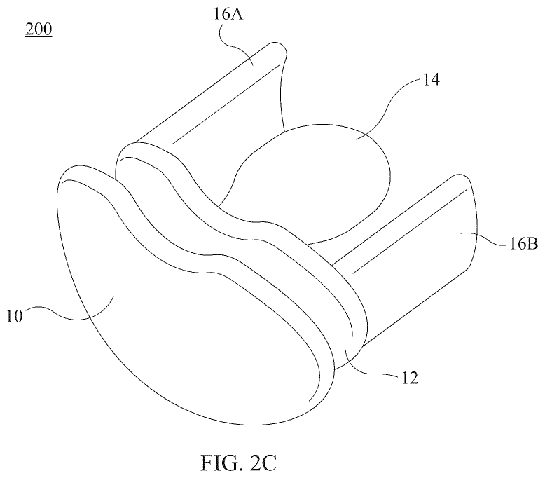

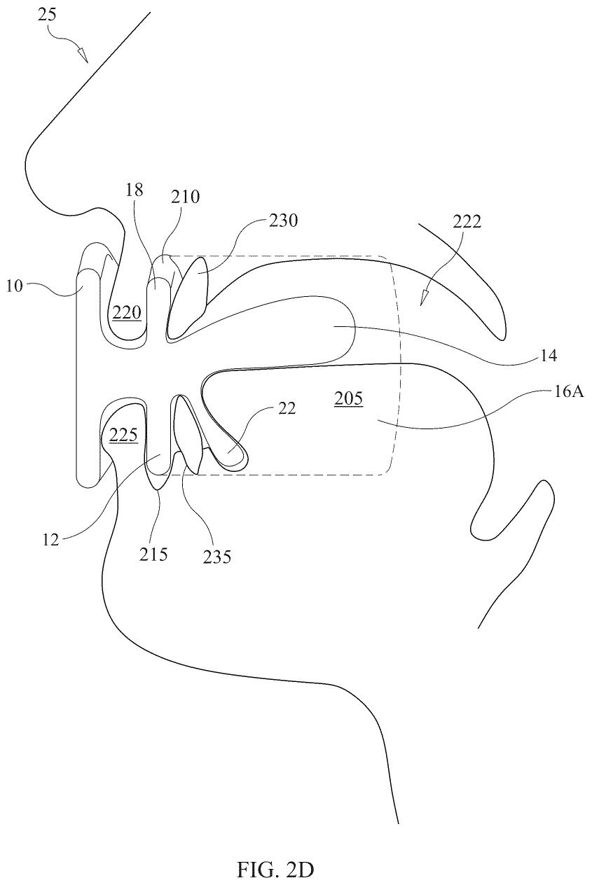

FIGS. 2A-2D are schematic diagrams illustrating various views of an exemplary mouth-cooling device 200, wherein FIG. 2A illustrates a top view; FIG. 2B illustrates a cross-section view; FIG. 2C illustrates a perspective view; and FIG. 2D illustrates a cross-section view a user 25 with mouth-cooling device 200 positioned within his mouth (i.e., a portion of mouth-cooling device 200 is positioned within the user's oral cavity 222 and a portion of mouth-cooling device 200 is positioned outside oral cavity 222). Mouth-cooling device 200 includes a superior oral vestibule portion 18 sized, positioned, and configured to be positioned proximate to and absorb heat from a user's superior oral vestibule 210 (i.e., between an interior side of the user's superior lip 220, superior oral vestibule 210, and superior teeth 230) during use as shown in FIG. 2D; an inferior oral vestibule portion 18 sized, positioned, and configured to be positioned proximate to and absorb heat from a user's inferior oral vestibule 215 (i.e., between an interior side of the user's inferior lip 225, inferior oral vestibule 215, and inferior teeth 235) during use as shown in FIG. 2D; an under-tongue extension 12 sized, positioned, and configured to be proximate to and absorb heat from a region in the user's mouth under the user's tongue 205 as shown in FIG. 2D, an over-tongue extension 14 sized, positioned, and configured to be proximate to and absorb heat from an upper side of the user's tongue 205 during use as shown in FIG. 2D, a left buccal extension 16A sized, positioned, and configured to be positioned proximate to and absorb heat from a left buccal region of the user's mouth during use as shown in FIG. 2D, a right buccal extension 16B sized, positioned, and configured to be positioned proximate to and absorb heat from a right buccal region of the user's mouth during use, and an external component 10 sized, positioned, and configured to reside outside the user's oral cavity and to be positioned proximate to the user's superior lip 220 and inferior lip 225 and cheeks during use as shown in FIG. 2D and hold mouth-cooling device 200 in place with the user's mouth when worn. In some embodiments, external component 10 may be configured to cool the user's superior lip 220, inferior lip 225, and cheeks, and/or exterior portions of the mouth, which may be helpful in preventing development of OM on the exterior parts of a user's lips, particularly among pediatric populations, who are particularly susceptible to the development of sores on the outside of their lips. External component 10 may also be configured to cool internal portions of the user's mouth from the outside in (e.g., superior oral vestibule 210 and inferior oral vestibule 215 are cooled by both outside component 10 and superior oral vestibule portion 18 and inferior oral vestibule portion 12, respectively.

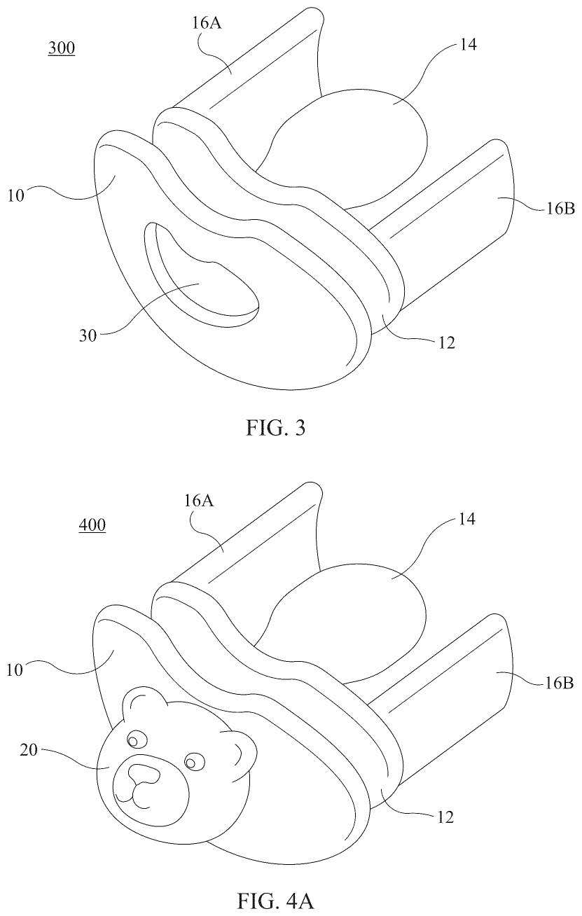

FIG. 3 provides a perspective view of another exemplary mouth-cooling device 300 that includes all the components of mouth-cooling device 200 and further includes an opening 30 sized, positioned, and configured to allows the user to breathe through the device while it is in use. Opening 30 may be a passageway through exterior component 10 so that it is open on both sides, thereby allowing the passage of air into, and out of, the user's mouth/oral cavity.

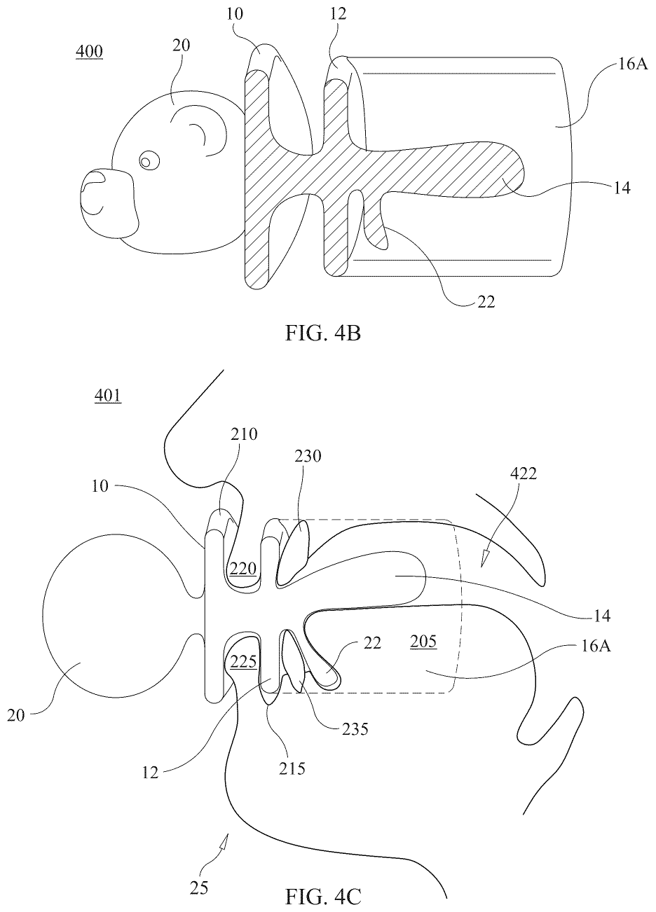

In some embodiments, the mouth-cooling devices disclosed herein may include an external extension that may be configured to be, for example, a reservoir of heat-absorbing material, a handle, and/or one or more decorative accents. FIG. 4A provides a top-perspective view of an exemplary mouth-cooling device with an external extension 400, FIG. 4B provides a side view of mouth-cooling device with external extension 400, and FIG. 4C is a sagittal cross-section view of a user with mouth-cooling device with external extension 400 positioned within his oral cavity. Mouth-cooling device with external extension 400 includes under-tongue extension 22, over-tongue extension 14, under-tongue extension 22, left buccal extension 16A, right buccal extension 16B, external component 10, superior oral vestibule portion 18, inferior oral vestibule portion 12, and an external extension 20. Mouth-cooling device with external extension 400 may be positioned within a user's 25 mouth in a manner substantially the same as the positioning of mouth-cooling device 200 within user's 25 mouth as shown in FIG. 2D. External extension 20 may be configured to be a handle that facilitates positioning of mouth-cooling device with external extension 400 within a user's mouth and/or keeping mouth-cooling device with external extension 400 in place within the user's mouth. Additionally, or alternatively, external extension 20 may be configured as a reservoir for storing heat-absorbing material. Additionally, or alternatively, external extension 20 may be configured to have a decorative feature (in this case, a teddy bear face (as shown in FIGS. 4A and 4B). Alternatively, external extension may have a round shape like external extension 20′ shown in FIG. 4C. In some embodiments, external extension 20 may be removable and/or exchangeable so that, for example, a plurality of external extensions 20 may be used with a single mouth-cooling device during, for example, a treatment session and/or to personalize the mouth-cooling device according to, for example, a user preference. For example, during a thirty minute treatment session, three external extensions 20 may be sequentially used (e.g., one external extension every 10 minutes) so that the cooling provided by the external extension is maintained throughout the treatment session.

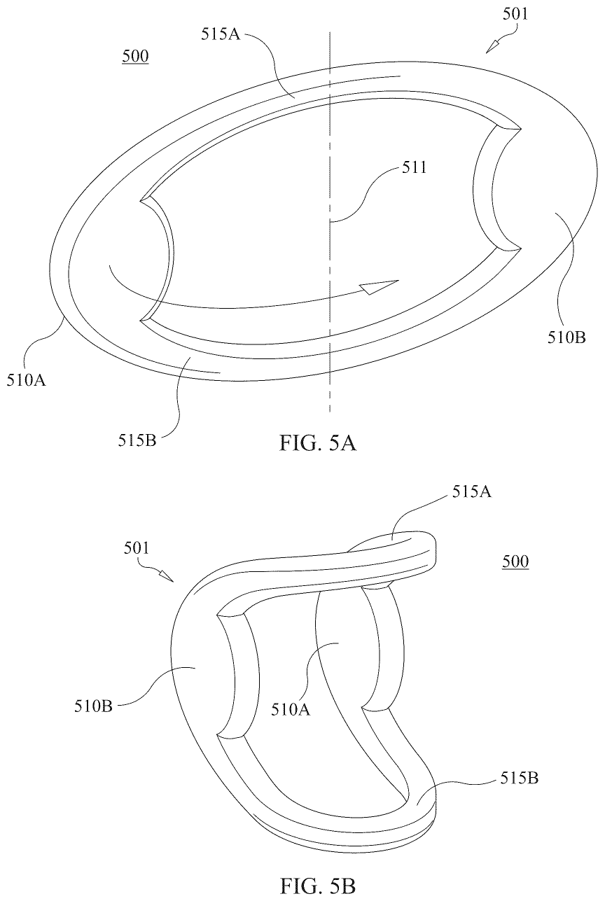

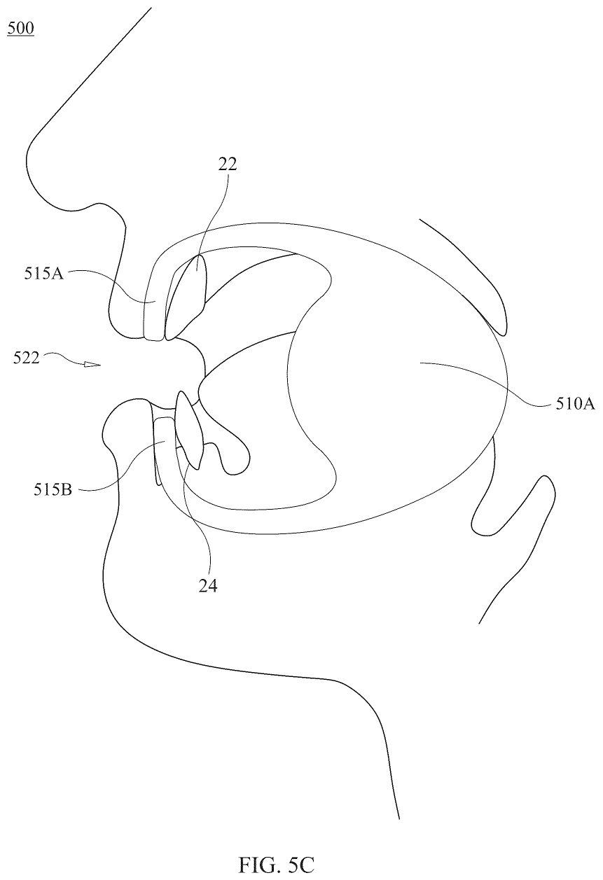

FIGS. 5A-5C show an embodiment of an elliptically-shaped mouth-cooling device 500 that includes a body 501 that holds a volume of heat-absorbing material therein. In particular, FIG. 5A is a top view of elliptically-shaped mouth-cooling device 500 when in an unfolded state; FIG. 5B is a side view of elliptically-shaped mouth-cooling device 500 when in a folded state; and FIG. 5C is a sagittal cross-section view of user 25 with folded elliptically-shaped mouth-cooling device 500 positioned within his oral cavity 522. Elliptically-shaped mouth-cooling device 500 includes a left buccal mucosa extension 510A, a right buccal mucosa extension 510B, a superior oral vestibule portion 515A, and an inferior oral vestibule portion 515B. FIG. 5A also includes a bisecting line 511 superimposed thereon that marks an approximate location about which elliptically-shaped mouth-cooling device 500 folds as shown in FIGS. 5B and 5C. As may be seen in FIG. 50, when elliptically-shaped mouth-cooling device 500 is bent (as shown in FIG. 5B) and positioned within a user's oral cavity 522 (as shown in FIG. 5C), right buccal mucosa extension 510B may be positioned proximate to the user's right buccal mucosa (e.g., between the user's teeth and cheek), superior oral vestibule portion 515A may be positioned proximate to the user's superior oral vestibule (e.g., between the user's superior lip and the gingiva of the superior jaw), and inferior oral vestibule portion 515B may be positioned proximate to the user's inferior oral vestibule (e.g., between the user's inferior lip and the gingiva of the inferior jaw).

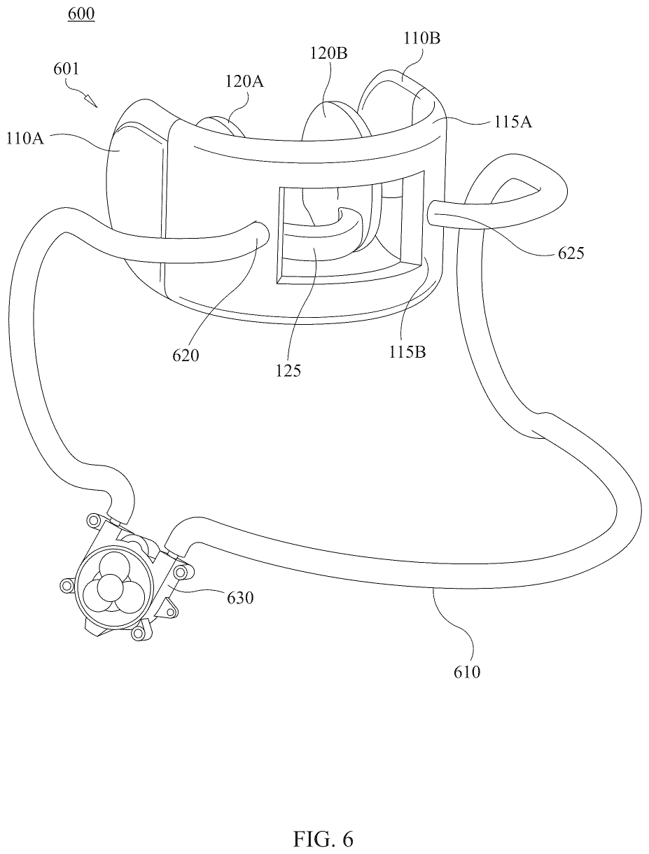

FIG. 6 provides a schematic diagram of an exemplary system 600 that includes a mouth-cooling device 601 similar mouth-cooling device 100 with the exception that it configured to couple to a tube 610 via an inlet port 620 and an outlet port 625. Tube 610 is coupled to a peristaltic pump 630, which pumps cooling vapor and/or liquid into mouth-cooling device 601 via inlet port 620 and pulls the cooling vapor/fluid through, and out of, mouth-cooling device 601 via outlet port 625.

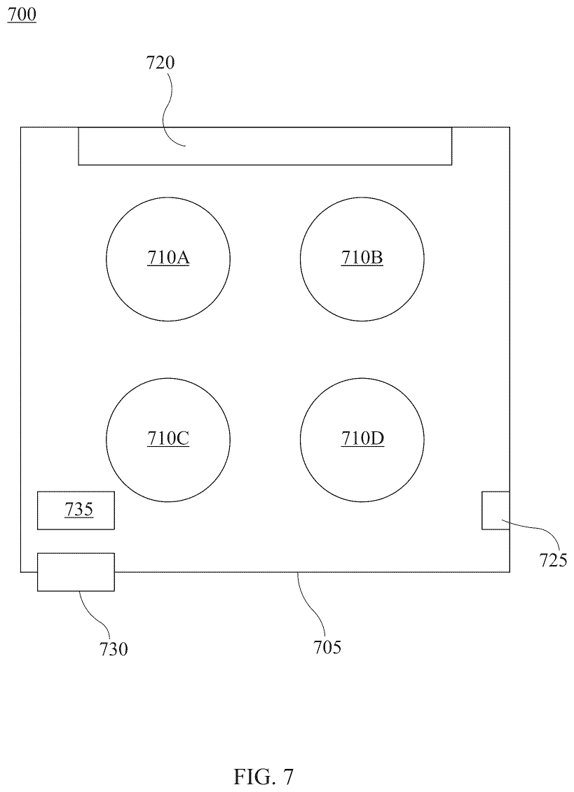

In some embodiments, the mouth-cooling devices disclosed herein may be configured for storage in a cooler and/or refrigerator and, while stored there, the heat-absorbing material and/or mouth-cooling device may achieve and/or be maintained at a desired temperature (e.g., 4-25 degrees Celsius). In these embodiments, one or more mouth-cooling devices may be provided in packaging (e.g., a sealed bag, box, and/or kit) for placement in the cooler and/or refrigerator. Additionally, or alternatively, a plurality of mouth-cooling devices may be packaged together as a kit 700, an example of which is provided by FIG. 7. Kit 700 includes a case 705 with a first, second, third, and fourth mouth-cooling device storage container, or recess, 710A, 710B, 710C, and 710D positioned therein. The first, second, third, and/or fourth recesses 710A, 710B, 710C, and/or 710D may be sized and configured to house one or more mouth-cooling devices like the mouth-cooling devices disclosed herein. In some embodiments, all of the mouth-cooling devices housed in case 705 may be of the same size and/or configuration and, in other embodiments, the mouth-cooling devices housed in case 705 may be of differing sizes and/or configurations. Case 705 may be made from a thermally insulating material (e.g., Styrofoam, plastic, and/or metal) so that it may prevent a heat exchange between the mouth-cooling devices (which may be pre-cooled) and an ambient environment.

In some embodiments, kit 700 may also include a refrigeration device 720 configured to cool the mouth-cooling devices to a desired temperature. In these embodiments, kit may further include a power source 730 and/or a temperature sensor (e.g., thermocouple and/or thermometer) 725 configured to sense a temperature within kit 700 and/or activate refrigeration device 720 responsively to a determination that the temperature is above a desired temperature. At times, kit 700 may further include a sanitizing kit 735 which may include, for example, sanitizing solution, a sanitization pouch configured for cooperation with an autoclave, and/or sanitizing wipes.

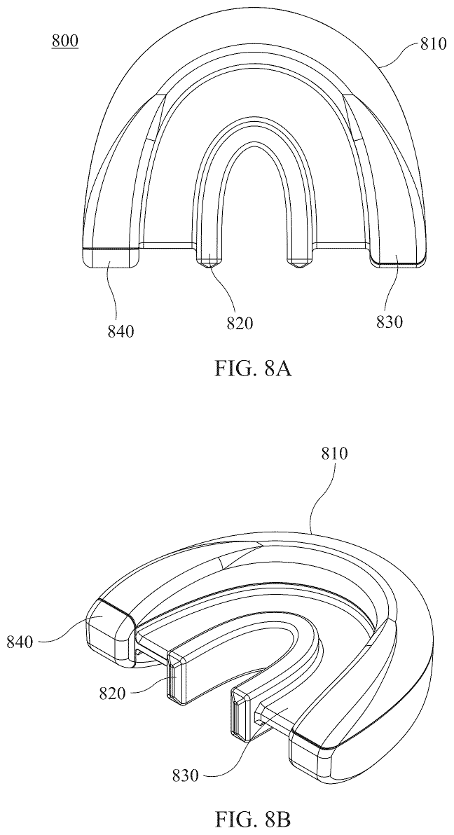

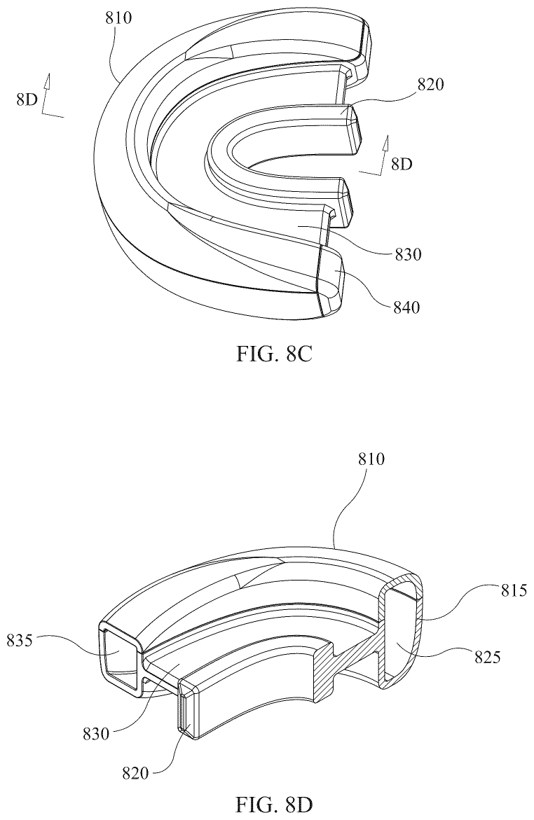

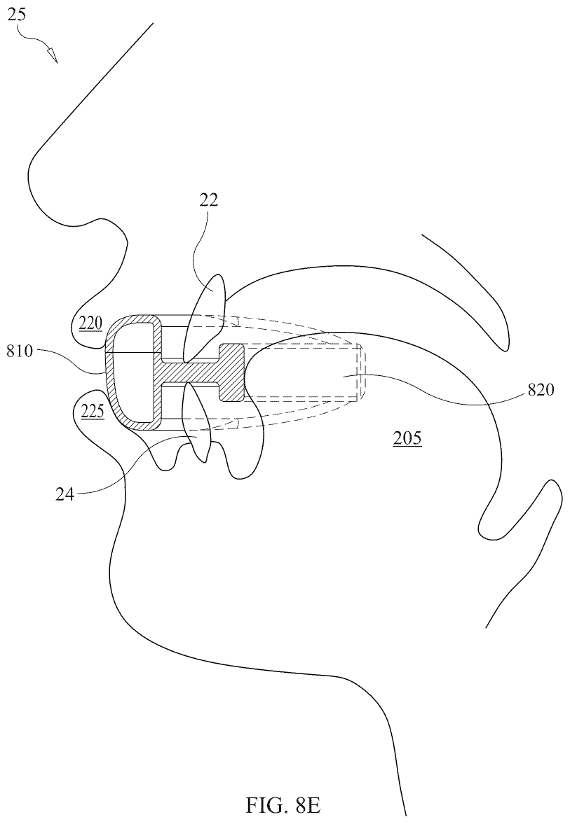

FIGS. 8A-8D are schematic diagrams that illustrate various views of another exemplary mouth-cooling device 800, wherein FIG. 8A provides a top view, FIG. 8B provides a back-perspective view, FIG. 8C provides a side-perspective view, and FIG. 8C provides a cross-section view of mouth-cooling device 800. Mouth-cooling device 800 includes an outer reservoir 810 including an outer reservoir cavity 815 (see in FIG. 8D), an inner reservoir 820 with an inner reservoir cavity 825, a connecting portion 830, an optional 835, and an optional plug 840. Outer reservoir 810 may be shaped to extend between a superior oral vestibule cavity of the user and an inferior vestibule cavity of the user when worn with a front side of the outer reservoir being configured to be placed proximate to the superior oral vestibule cavity and inferior oral vestibule cavity and a back side being configured to be proximate to a buccal side of the user's upper jaw and lower jaw when worn by the user as shown in, for example, FIG. 8E.

Inner reservoir 820 may be shaped and/or configured to extend between a lingual side of the user's upper jaw and lower jaw and abut the user's tongue when worn by the user as shown in, for example, FIG. 8E. Connecting portion 830 may be shaped and configured to connect the inner reservoir and the outer reservoir and fit between the user's upper jaw and lower jaw when worn. When the user has teeth, connecting portion 830 may be positioned between the user's upper and lower teeth as shown in, for example, FIG. 8E. In some embodiments, connecting portion 830 may be solid (i.e., not include a cavity) so that it does not cool the teeth positioned proximate thereto.

Mouth-cooling device 800 may further include a heat-absorbing material positioned within outer reservoir cavity 815 and inner reservoir cavity 825 and the heat-absorbing material may be configured to absorb heat from inside and outside the user's mouth, lips, and/or cheeks when mouth-cooling device 800 is positioned within the user's mouth as shown in, for example, FIG. 8E. Mouth-cooling device 800 may further include optional opening 835 sized and configured to accept insertion of optional plug 840 therein. Plug 840 may be sized and configured fit within opening 835 and form a water-tight seal (via, for example, friction and/or mechanical sealing), thereby sealing the heat-absorbing material within at least one of the outer reservoir cavity and the inner reservoir cavity. In some embodiments, heat-absorbing material may be removed from and/or added to mouth-cooling device 800 via removal of plug 840 from opening 835 in order to, for example, remove heat-absorbing material that has absorbed heat from the user's mouth and replace the warmed heat-absorbing material with cool, fresh, heat-absorbing material.

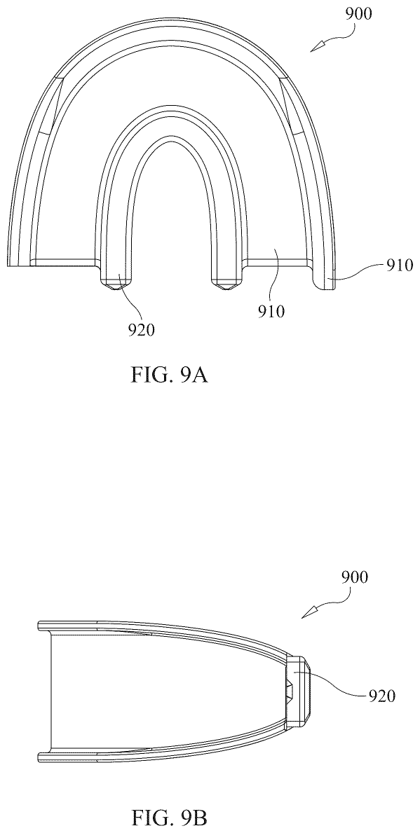

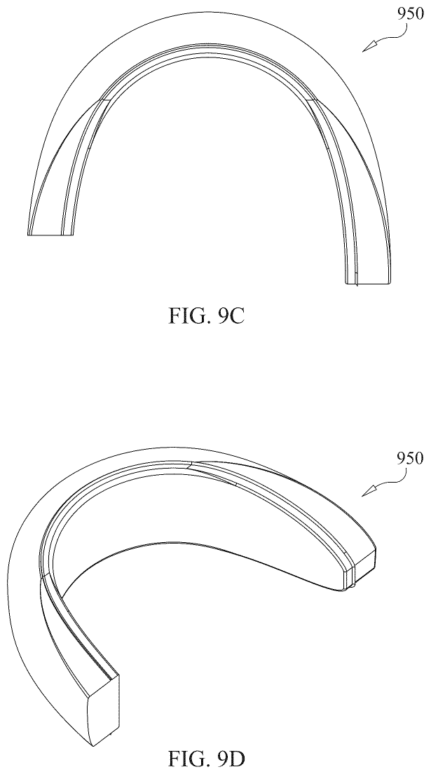

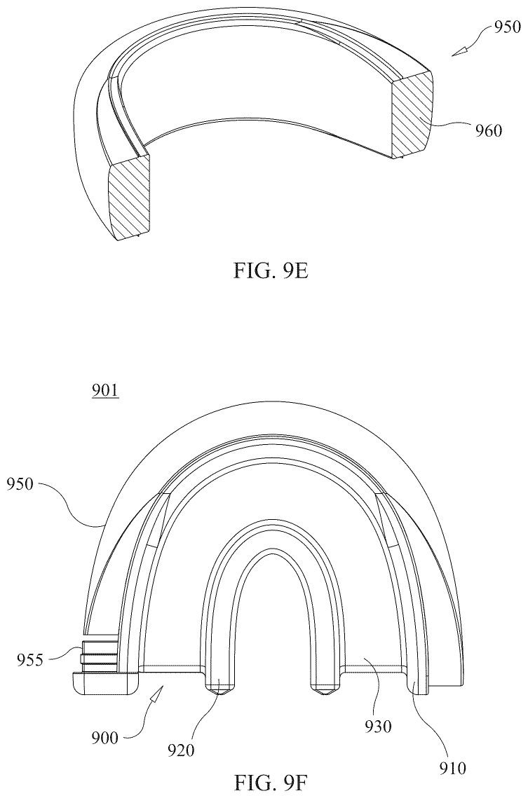

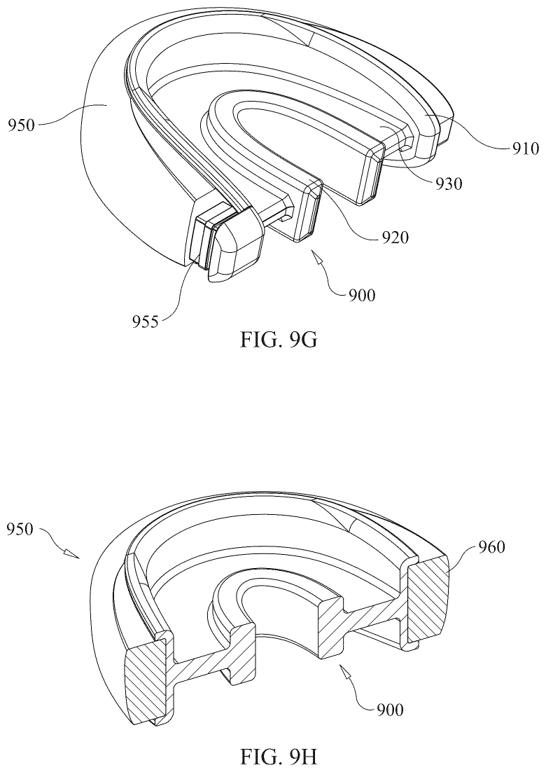

FIGS. 9A-9H provide various views an assembled mouth-cooling device 901 and components thereof. In particular, FIGS. 9A and 9B are schematic diagrams illustrating top and side views, respectively, of a base 900; FIGS. 9C-9E are schematic diagrams illustrating top, perspective, and cross-section views, respectively, of a cooling attachment 950, and FIGS. 9F, 9G, and 9H are top, rear-perspective, and cross-section views, respectively, of an assembly 901 of base 900 and cooling attachment 950. Base 900 includes an outer portion 910, an inner reservoir 920 including an inner reservoir cavity like inner reservoir cavity 925 and connecting portion. An exterior edge of outer portion 910 includes one or more attachment mechanisms configured to attach to cooling attachment as shown in the cross-section view of FIG. 9H. As may be seen in FIG. 9H, cooling attachment 950 may fit into a corresponding notch or indentation in an exterior edge of base 900; the indentation being sized and configured to removably accept insertion of cooling attachment 950 and be held in place therein via, for example, friction and/or bonding (e.g., glue, sealant, etc.).

Cooling attachment 950 is sized and shaped to cooperate with an outer edge of body 900 and attach thereto as shown in, for example, FIGS. 9F-9H. Cooling attachment 950 is shaped to extend between a superior oral vestibule cavity of the user and an inferior vestibule cavity of the user when worn with a front side of the outer reservoir being configured to be placed proximate to the superior oral vestibule cavity and inferior oral vestibule of the user in a manner similar to that shown in FIG. 8E. Cooling attachment 950 includes a cooling attachment cavity 960 shaped and configured for acceptance of heat-absorbing material therein via, for example, removal of a plug 955 from a corresponding opening in cooling attachment 950.

In some embodiments, assembly 901 may be worn by a user to cool the user's mouth and, when the heat-absorbing material is no longer sufficiently cooling the mouth (e.g., has reached a temperature above 20 or 25 degrees Celsius), assembly 901 may be removed from the user's mouth, a first cooling attachment 950 may be removed from body 900 and a second, fresh and cooled (e.g., between 5-20 degrees Celsius) cooling attachment 950 may be attached to body 900 and the new assembly 901 may be inserted into the user's mouth for continued cooling thereof.

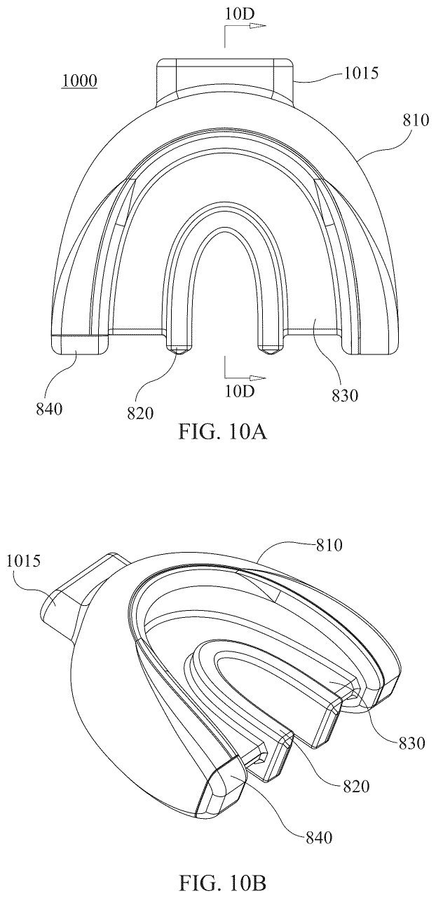

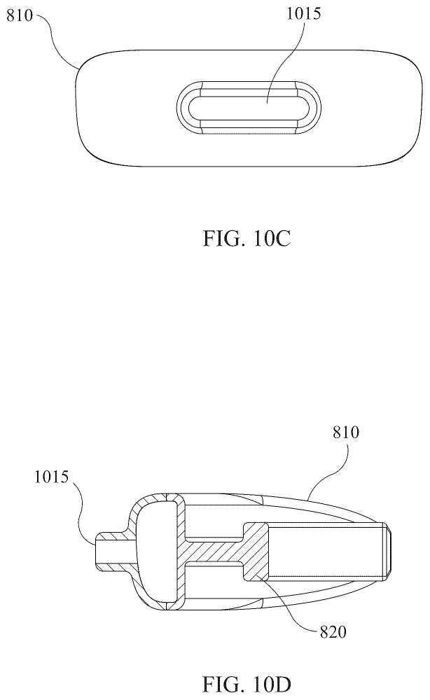

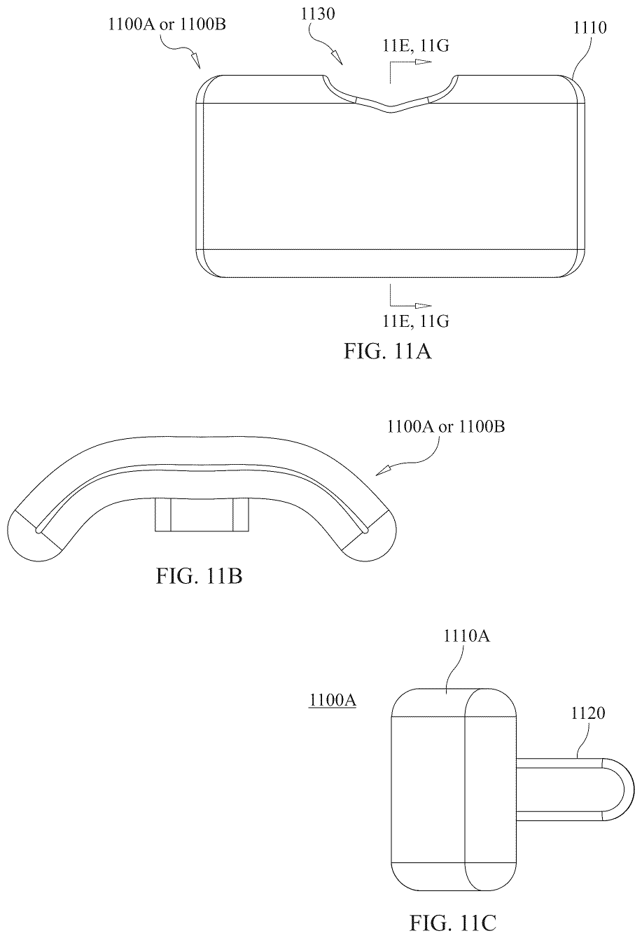

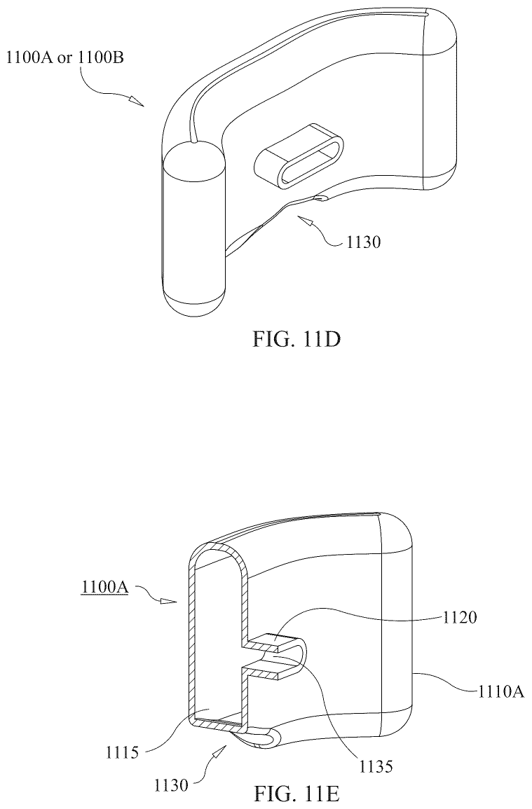

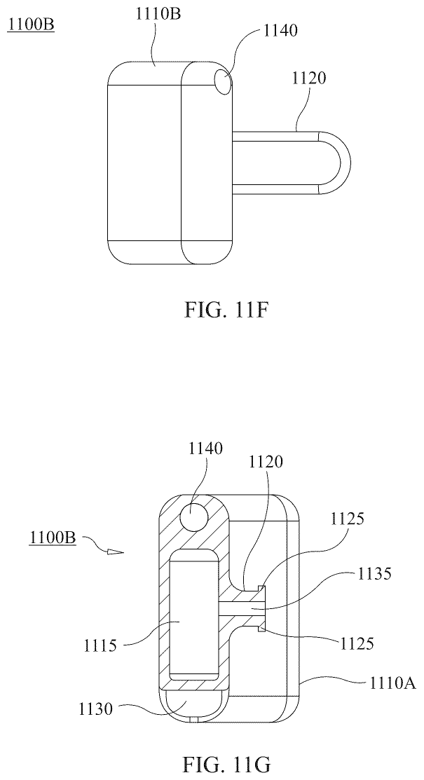

FIGS. 10A-10D are schematic diagrams that illustrate various views of another exemplary mouth-cooling device body 1000, wherein FIG. 10A provides a top view, FIG. 10B provides perspective view, FIG. 10C provides a front view, and FIG. 10D provides a cross-section view thereof. Mouth-cooling device 1000 is similar to mouth-cooling device 800 because it includes outer reservoir 810, which includes outer reservoir cavity 815, inner reservoir 820, which includes inner reservoir cavity 825, connecting portion 830, optional 835, and optional plug 840. In addition to these components, mouth-cooling device body 100 further includes an attachment coupling 1015 configured for attachment to an external component like external component 1100 shown in FIGS. 11A-11D.

FIGS. 11A-11G are schematic diagrams that illustrate various views of an exemplary fixed-shape exterior component 1100A and/or an exemplary deformable exterior component 1100B, wherein FIG. 11A is a front view, FIG. 11B is a top view, FIG. 11C is a side view, FIG. 11D is a rear-perspective view of fixed-shape exterior component 1100A and/or deformable exterior component 1100B; FIG. 11E is a cross-section view of fixed-shape exterior component 1100A; FIG. 11F is a side view of deformable exterior component 1100B; and FIG. 11G is a cross-section view of deformable exterior component 1100B. An upper (as oriented in FIG. 11A) edge of fixed-shape exterior component 1100A and/or deformable-shape exterior component 1100B may include an optional notch 1130 present in an approximate middle of the upper edge. Notch 1130 may be configured to allow for external component to extend between the user's lips and nose but not touch or overly the user's nose as shown in, for example, 15A-15C.

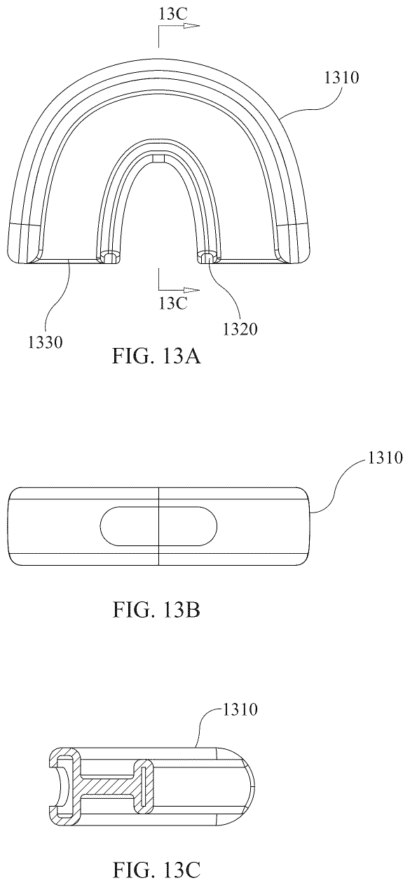

Fixed-shape exterior component 1100A includes a fixed-shape body 1110A from which an attachment extension 1120 extends as shown in FIGS. 11C-11E. Fixed-shape body 1110A also includes a cavity 1115 (as shown in the cross-section of FIG. 11E) within which heat-absorbing material may be resident. Extension 1120 may include a central lumen, or channel, 1135 through which heat-absorbing media may be put into and/or extracted from cavity 1115 when, for example, adding cooled heat-absorbing material to cavity 1115 and/or transferring heat-absorbing media to and/or from a cavity in a corresponding mouth-cooling device body as shown in, for example, FIGS. 13A-13F and discussed below. Extension 1120 may further include an upper (as oriented in FIG. 11E) and a lower tab 1125 configured to engage with a corresponding opening of a mouth-cooling device body, thereby holding fixed-shape exterior component 1100A and the mouth-cooling device body together as shown in, for example, FIGS. 13A-13F and discussed below.

Fixed-shape body 1110A may be shaped and configured so that it resides outside the user's mouth when a mouth-cooling device is worn and is curved to extend from approximately a left commissure of the user's lips to a right commissure of the user's lips and/or extend between the user's left and right buccal mucosa extensions when a mouth-cooling device is worn. An interior arc of curvature for body 1110 may be curved at a rate of 5 mm per 30 mm of length so that an apex on the interior arc is 25 mm away from a lowest point of the arc as shown in the example of FIG. 11B. Approximate dimensions for body 1110 range between 40-80 mm high, 80-175 mm wide, and 12-30 mm thick with the dimensions of one embodiment of mouth-cooling device body 1110 being 65 mm high by 125 mm wide by 20 mm thick.

Deformable exterior component 1100B is substantially similar to fixed-shape exterior component 1100A with the exception that it includes a deformable element 1140 configured and positioned within deformable body 1110B so that it may be deformed and hold a deformed shape, which thereby allows deformable body 1110 to be bent, or deformed, to for example, conform to anatomical features of a user's face. Deformable element 1140 may be, for example, a wire, a shape-memory wire, a compressible foam, or any other component configured to deform and hold its deformed shape.

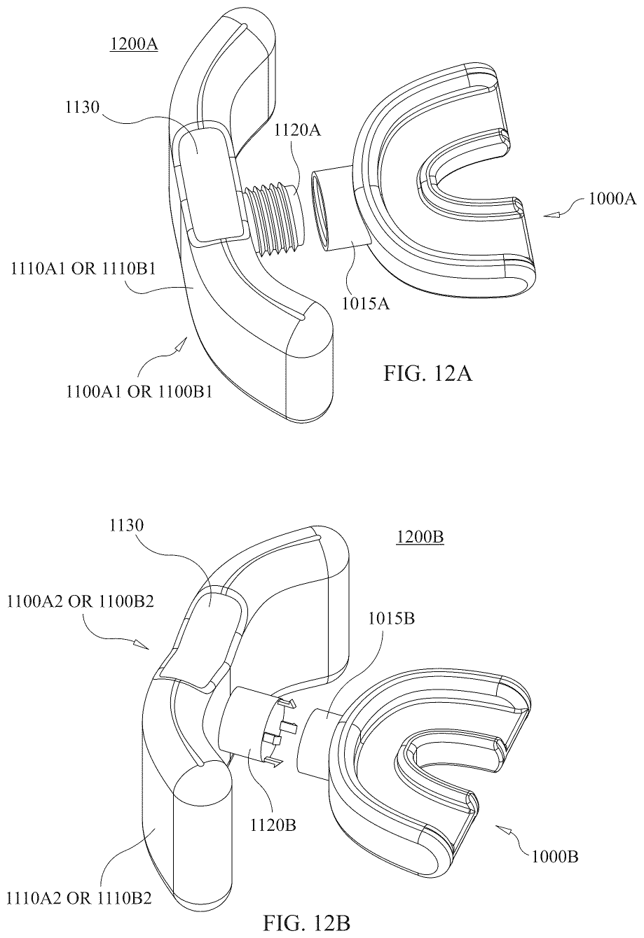

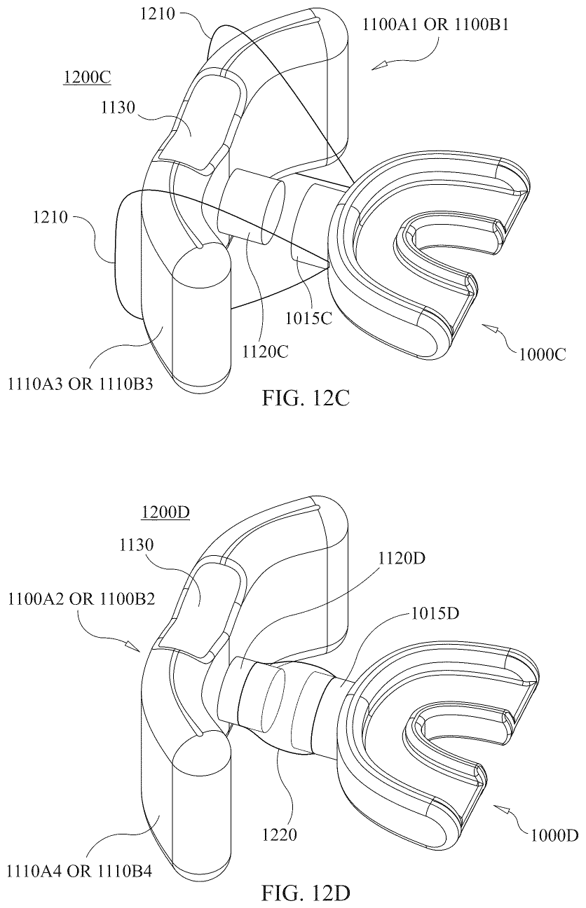

FIGS. 12A-12D provide schematic diagrams of exploded views of four different mouth-cooling devices that include a mouth-cooling device body 1000 and four different embodiments of external component 1100A and 1100B, wherein FIG. 12A provides an exploded view of a first mouth-cooling device 1200A that includes a first mouth-cooling body 1000A with a first attachment coupling 1015A comprising a series of threads sized, shaped, and configured to cooperate with and engage corresponding threads on a first attachment extension 1120A that extends from a first fixed-shape body 1110A1 and/or a first deformable body 1110B1 of a first fixed-shape external component 1100A1 and/or a first deformable external component 1100B1 as shown. First mouth-cooling device body 1000A and first fixed-shape external component 1100A1 and/or a first deformable external component 1100B1 may be assembled together by aligning first attachment extension 1120A with first attachment coupling 1015A and twisting first attachment extension 1120A relative to first attachment coupling 1015A in a screw-like manner so that first fixed-shape body 1110A1 and/or a first deformable body 1110B1 is removably attached to first mouth-cooling device body 1000A.

FIG. 12B provides an exploded view of a second mouth-cooling device 1200B that includes a second mouth-cooling body 1000B with a second attachment coupling 1015B that includes a plurality of cantilever and/or u-shaped snap fit joints sized, shaped, and configured to cooperate with and engage with a corresponding mechanism (e.g., a snap-fit joint) of a second attachment extension 1120B of a second fixed-shape body 1110A2 and/or a second deformable body 1110B2 of a respective second fixed-shape external component 1100A2 and/or second deformable external component 1100B2 as shown. Second mouth-cooling device body 1000B and second fixed-shape body 1110A2 and/or a second deformable body 1110B2 may be assembled together by aligning second attachment extension 1120B with second attachment coupling 1015B and pressing them together so that second fixed-shape body 1110A2 and/or a second deformable body 1110B is removably attached to second mouth-cooling device body 1000B, thereby assembling second mouth-cooling device 1200B.

FIG. 12C provides an exploded view of a third mouth-cooling device 1200C that includes a third mouth-cooling body 1000C with a third attachment coupling 1015C and a third fixed-shape body 1110A3 and/or a third deformable body 1110B3. Third mouth-cooling body 1000C includes a third attachment coupling 1015C with an opening therein sized, shaped, and configured to cooperate with and engage with a corresponding mechanism of a third attachment extension 1120C of a third fixed-shape body 1110A3 and/or a third deformable body 1110B3 as shown. Third mouth-cooling device body 1000C and third fixed-shape external component 1100A3 and/or third deformable external component 1100B3 may be assembled together by aligning third attachment extension 1120C with third attachment coupling 1015C and pressing them together so that third external component 1100C is removably attached to third mouth-cooling device body 1000C. One or more retention mechanisms 1210 that extend between third mouth-cooling device body 1000C and third fixed-shape body 1110A3 and/or a third deformable body 1110B3 may then be put in place as shown to secure assembly of third mouth-cooling device 1204.

FIG. 12D provides an exploded view of a fourth mouth-cooling device 1200D that includes a fourth mouth-cooling body 1000D with a fourth attachment coupling 1015D with an opening therein sized, shaped, and configured to cooperate with and engage with a corresponding mechanism of a fourth attachment extension 1120D of a fourth fixed-shape body 1110A4 and/or a fourth deformable body 1110B4. Fourth mouth-cooling device body 1000D and fourth fixed-shape body 1110A4 and/or a fourth deformable body 1110B4 may be assembled together by aligning fourth attachment extension 1120D with fourth attachment coupling 1015D and pressing them together so that fourth fixed-shape body 1110A4 and/or a fourth deformable body 1110B4 is removably attached to fourth mouth-cooling device body 1000D. A sleeve 1220 may then be placed over a joint between fourth attachment coupling 1015D and fourth attachment extension 1120D as shown to secure assembly of fourth mouth-cooling device 1204.

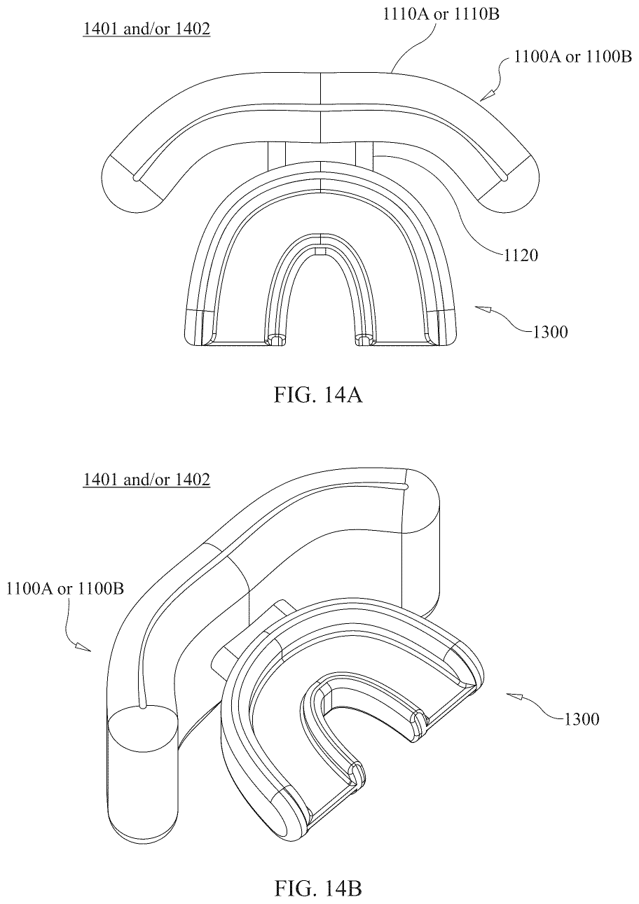

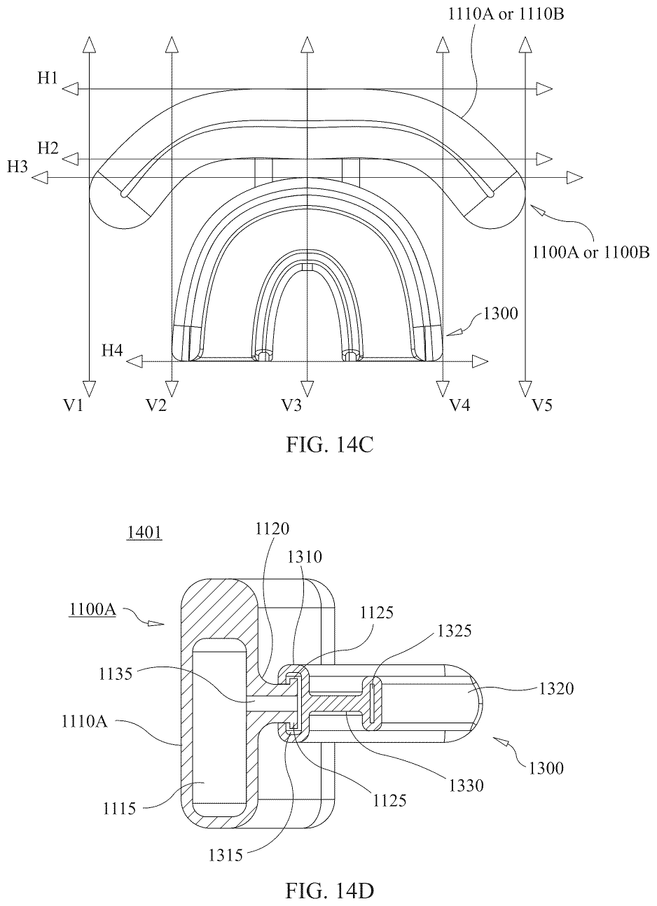

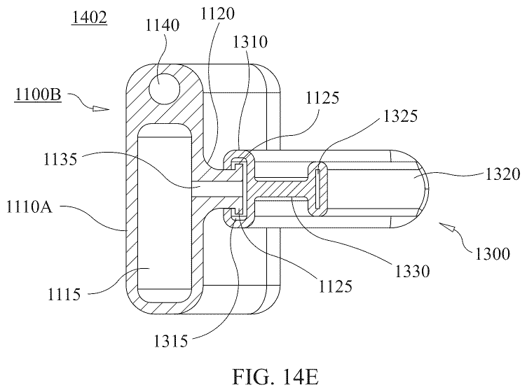

FIGS. 13A-13C provide schematic diagrams illustrating various views of another exemplary mouth-cooling device 1300, wherein FIG. 13A provides a top view, FIG. 13B provides a front view, and FIG. 13C provides a cross-section view of mouth-cooling device 1300. Mouth-cooling device 1300 is similar to mouth-cooling device 800 in that it comprises an outer reservoir 1310 including an outer reservoir cavity 1315 (see in FIG. 13C), an inner reservoir 1320 with an inner reservoir cavity 1325 (see FIG. 13C), and a connecting portion 1330. Unlike mouth-cooling device 800, mouth-cooling device 1300 also includes an opening 1350 (see FIG. 13B) into which attachment extension 1120 may be inserted as shown in, for example, FIGS. 14A-14D. Heat-absorbing media may be resident within inner reservoir cavity 1325 and/or outer reservoir cavity 1315.

FIGS. 14A-14E provide schematic diagrams illustrating various views of an assembly 1401 of mouth-cooling device 1300 and fixed-shape body 1110A (FIGS. 14A-1DC) and/or an assembly 1402 of mouth-cooling device 1300 and deformable body 1110B (FIGS. 14A, 14B, 14C, and 14E), wherein FIG. 14A provides a top view and FIG. 14B provides a perspective view of assembly 1401 and/or 1402 and FIG. 14C provides a top view of assembly 1401 and/or 1402 with an arrangement of reference lines superimposed thereon, FIG. 14D provides a cross-section view of assembly 1401, and FIG. 14E provides a cross-section view of assembly 1402.

As may be seen in the cross-section views FIGS. 14D and 14E, when assembled, attachment extension 1120 of fixed-shape body 1110A and/or deformable body 1110B are inserted into opening 1350 so that upper and lower tabs 1125 are deformed to push through opening 1350 and then return to their original shape once inside outer reservoir 1315, which holds fixed-shape body 1110A or deformable body 1110B in place relative to mouth-cooling device 1300. Cavity 1115 and outer reservoir 1315 may be in fluid communication with one another via channel 1135 whereby heat-absorbing material resident within cavity 1115 may be added to outer reservoir 1315 and/or exchanged with heat-absorbing material resident within outer reservoir 1315.

The gridlines of FIG. 14C provide exemplary dimensions for assembly 1401 and/or 1402 wherein there are four horizontal reference lines: H1, H2, H3, and H4 and four vertical reference lines: V1, V2, V3, and V4. Approximate distances between these reference lines are included in Table 1 as follows:

TABLE 1

| Reference Lines | Distance between reference lines in mm |
|---|---|
| H1 to H2 | 20 mm ± 2 mm |
| H2 to H3 | 6.6 mm ± 0.5 mm |
| H3 to H4 | 52 mm ± 2.5 mm |
| V1 to V2 | 12.5 mm ± 0.7 mm |
| V2 to V3 | 38.8 mm + 2 mm |
| V3 to V4 | 38.8 mm ± 2 mm |

These distances of Table 1 are exemplary and may be proportionally scaled up or down to make an assembly and/or mouth-cooling device that is sized to fit a particular user. In some instances, they may be scaled and/or proportioned in pre-set sizes such as small, medium, and large; adult, child, and infant; adjusted to account for skin thickness, etc.

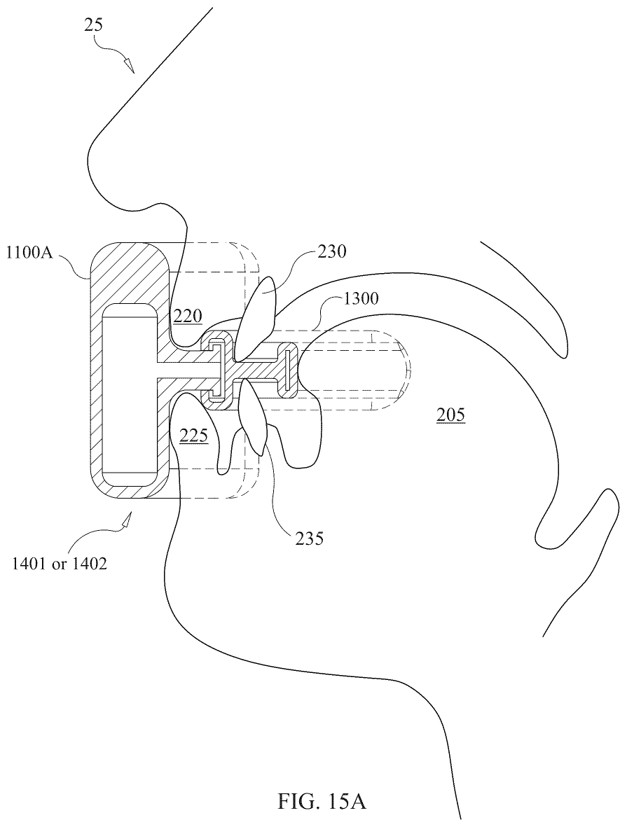

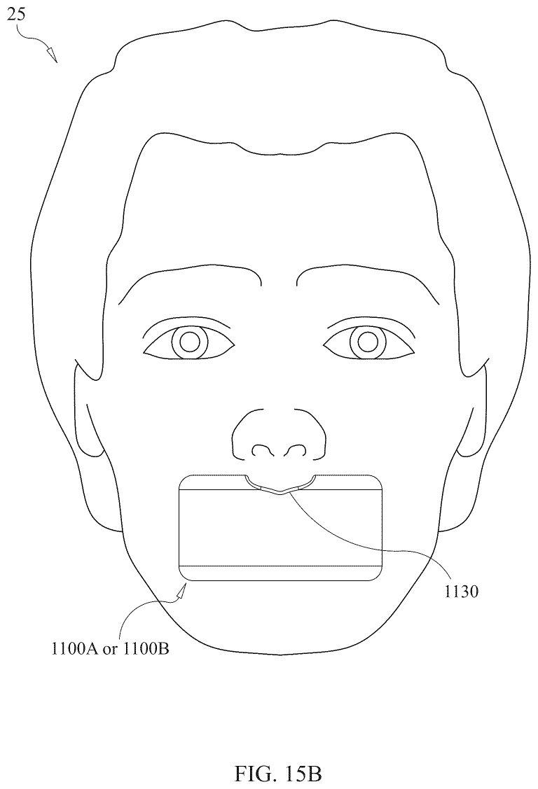

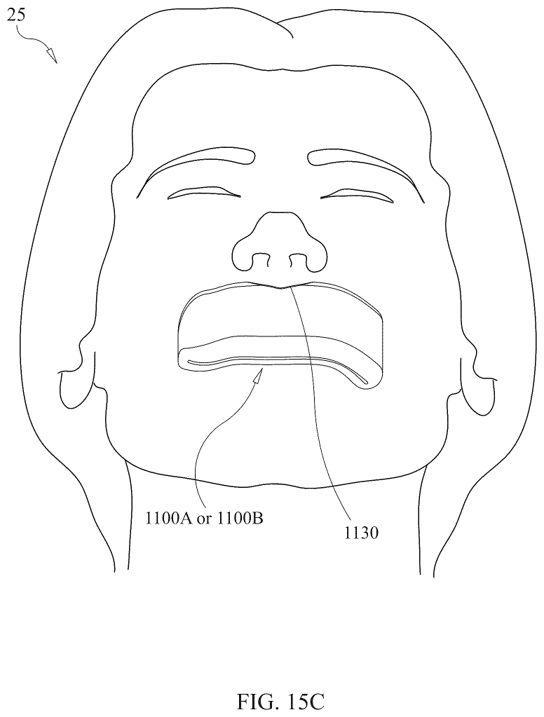

FIG. 15A is a schematic diagram illustrating a mid-sagittal cross-section view, FIG. 15B is a front view, and FIG. 15C is a bottom-perspective view of user 25 with the mouth-cooling device of 14A correctly positioned (i.e., being worn) in his anatomy so that it is proximate to user's 25 mouth and surrounding tissue (e.g., lips, skin, cheeks, etc.). As may be seen in FIG. 15A, a right side of mouth-cooling device 1300 is in position proximate to a right side of user's 25 tongue and a right side of fixed-shape exterior component 1100A or deformable exterior component 1100B wraps around the user's 25 face so that it covers the lips and skin covering buccal side of the user's upper jaw and lower jaw. As may be seen in FIGS. 15B and 15C, fixed-shape exterior component 1100A or deformable exterior component 1100B resides outside the user's 25 mouth when the mouth-cooling device is worn and extends from approximately a left commissure of the user's lips to approximately a right commissure of the user's lips.

1. A mouth-cooling device comprising:
a body configured to fit within a user's mouth, the body comprising:
an outer reservoir including an outer reservoir cavity, the outer reservoir being shaped to extend between a superior oral vestibule cavity of the user and an inferior vestibule cavity of the user when worn with a front side of the outer reservoir being configured to be placed proximate to the superior oral vestibule cavity and the inferior oral vestibule cavity and a back side being configured to be proximate to a buccal side of the user's upper jaw and lower jaw when worn by the user;
an inner reservoir including an inner reservoir cavity, the inner reservoir being shaped to extend between a lingual side of the user's upper jaw and a lingual side of the user's lower jaw and abut the user's tongue when worn;
a connecting portion configured to connect the inner reservoir and the outer reservoir and fit between the user's upper jaw and lower jaw when worn;
an exterior component that includes an external cavity, the exterior component being sized and configured to reside outside the user's mouth when the mouth-cooling device is worn and extend from approximately a left commissure of the user's lips to approximately a right commissure of the user's lips, wherein a volume of the heat-absorbing material is resident within the external cavity; and
a heat-absorbing material resident within the outer reservoir cavity, inner reservoir cavity, and the exterior component cavity, the heat-absorbing material being configured to absorb heat from inside and outside the user's mouth when the mouth-cooling device is positioned therein.

2. The mouth-cooling device of claim 1, wherein the mouth-cooling device is configured to reduce capillary blood flow within the user's mouth, lips, and/or cheeks when worn.

3. The mouth-cooling device of claim 1 or 2, wherein the mouth-cooling device is configured to induce vasoconstriction within the user's mouth, lips, and/or cheeks when worn.

4. The mouth-cooling device of claim 1, 2, or 3, wherein the mouth-cooling device is configured to reduce a metabolism rate of cells within the user's mouth, lips, and/or cheeks when worn.

5. The mouth-cooling device of claim 4, wherein the cells are oral epithelial stem cells.

6. The mouth-cooling device of claim 1, wherein the body and heat-absorbing material are configured to prevent or mitigate development of oral mucositis for the user.

7. The mouth-cooling device of claim 1, wherein the body and heat-absorbing material are configured to prevent oral mucositis for the user before, during, or after the user receives chemotherapy treatment and/or radiotherapy treatment.

8. The mouth-cooling device of claim 1, wherein a portion of the mouth-cooling device is malleable.

9. The mouth-cooling device of claim 1, wherein a portion of the mouth-cooling device is configured to deform to be proximate to the user's mouth, lips, or skin.

10. The mouth-cooling device of claim 1, wherein a portion of the mouth-cooling device is configured to deform thereby conforming to user anatomy.

11. The mouth-cooling device of claim 1, further comprising an over-tongue extension with an over-tongue extension cavity, the over-tongue extension extending from an inner side of the inner reservoir and being sized and configured to reside between the user's tongue and mouth roof when worn and the extension cavity being filled with heat-absorbing material configured to absorb heat from the user's tongue and mouth roof when the body is positioned within the user's mouth.

12. The mouth-cooling device of claim 1, further comprising an extension with an extension cavity, the extension extending from an inner side of the inner reservoir and being sized and configured to reside under the user's tongue when the body is positioned within the user's mouth.

13. The mouth-cooling device of claim 1, wherein the body includes an opening sized and configured to accept insertion of a plug therein, wherein the plug seals the heat-absorbing material within at least one of the outer reservoir cavity and the inner reservoir cavity.

14. The mouth-cooling device of claim 13, wherein a volume of heat-absorbing material may be removed from and/or added to at least one of the outer reservoir cavity and the inner reservoir cavity via removal of the plug from the opening.

15. The mouth-cooling device of claim 1, wherein the exterior component is configured to extend between a left buccal mucosa extension and a right buccal mucosa extension of the user's mouth when the body is positioned within the user's mouth.

16. The mouth-cooling device of claim 1, wherein the exterior component is configured to extend between and beyond a left buccal mucosa extension and a right buccal mucosa extension of the user's mouth when the body is positioned within the user's mouth.

17. The mouth-cooling device of claim 1, wherein the exterior component is configured to extend between a left commissure of the user's mouth and a right commissure of the user's mouth when the body is positioned within the user's mouth.

18. The mouth-cooling device of claim 1, wherein the exterior component is configured to extend between and beyond a left commissure of the user's mouth and a right commissure of the user's mouth when the body is positioned within the user's mouth.

19-50. (canceled)

51. A method comprising:
placing the body of the mouth-cooling device of claim 1 within the mouth of a user; and
positioning the exterior portion of any of the mouth-cooling devices of claims 1-43 outside the user's mouth proximate to the user's lips so that the body and the exterior portion absorb heat from the user's mouth, lips, and/or cheeks, reduce capillary blood flow within the user's mouth, lips, and/or cheeks, induce vasoconstriction within the user's mouth or lips, reduce a metabolism rate of cells within the user's mouth, lips, and/or cheeks, or prevent or mitigate development of oral mucositis for the user.

52-55. (canceled)

56. A mouth-cooling device comprising:
a base configured to fit within a user's mouth, the comprising:
an outer edge configured to be removably coupled to a cooling attachment;
an inner reservoir including an inner reservoir cavity, the inner reservoir being shaped to extend between a lingual side of the user's upper jaw and lower jaw and abut the user's tongue when worn;
a connecting portion configured to connect the inner reservoir and the outer reservoir and fit between the user's upper jaw and lower jaw when worn;
the cooling attachment removably coupled to the outer edge of the body, the cooling attachment including a cooling attachment cavity and being shaped to extend between a superior oral vestibule cavity of the user and an inferior vestibule cavity of the user when worn with a front side of the outer reservoir being configured to be placed proximate to the superior oral vestibule cavity and inferior oral vestibule cavity; and
a heat-absorbing material resident within inner reservoir cavity and the cooling attachment cavity, the heat-absorbing material being configured to absorb heat from inside and outside the user's mouth when the mouth-cooling device is positioned therein.

57-58. (canceled)
